(19) United States
(12) Patent Application Publication
(10) Pub. No.: US 2025/0193977 A1
(43) Pub. Date: Jun. 12, 2025

(54) COMPOSITE LIGHT EMISSION SIGNAL GENERATION MATERIAL FOR STATE SENSING, LIGHT-EMITTING SUBSTANCE CARRIER, INK FOR STATE SENSING, MEASUREMENT CHIP, AND ANALYSIS METHOD

(71) Applicant: Konica Minolta, Inc., Tokyo (JP)

(72) Inventors: Shumpei HITOSUGI, Tokyo (JP); Hiroshi KITA, Tokyo (JP)

(21) Appl. No.: 18/845,049

(22) PCT Filed: Mar. 9, 2023
(86) PCT No.: PCT/JP2023/009004

§ 371 Date: Sep. 9, 2024

(30) Foreign Application Priority Data
Mar. 10, 2022 (JP) 2022-037179
Dec. 19, 2022 (JP) 2022-202044

(51) Int. Cl. H05B 33/14; C07H 21/04; C09K 11/06; G01N 21/64; H05B 33/20

(52) U.S. Cl. CPC H05B 33/14; C07H 21/04; C09K 11/06; G01N 21/645; H05B 33/20; G01N 2021/6482

(57) ABSTRACT

Provide a composite luminescent signal generating material for state sensing which easily interacts with a target substance and can acquire a large amount of date. A composite luminescent signal generating material for state sensing that solves the above-described problems includes a nucleic acid structure and at least one luminescent compound residue bonded to a main chain of the nucleic acid structure, and emits, for a single excitation light, two or more types of light selected from the group consisting of fluorescence, phosphorescence, excimer light emission, exciplex light emission, thermally activated delayed fluorescence, excited state intramolecular proton emission, triplet triplet annihilation emission, twisted intramolecular charge transfer emission, and aggregation-induced emission.

### DESCRIPTION

### Technological Field

The present invention relates to a composite luminescent signal generating material for state sensing, a luminescent substance carrier, an ink for state sensing, a measurement chip, and an analysis method.

### Description of Related Art

Conventionally, as a molecular probe for analyzing various target substances, it has been required that the molecular probe specifically interacts with the target substance, and furthermore, the interaction with the target substance is easy for an observer to understand. For example, substances that specifically react with mercury ions to emit light, substances that specifically react with pH to change the color, and the like have been used as molecular probes. For example, it has been proposed to use, for various analyses, molecular probes in which a luminophore or a chromophore is bonded to a main chain containing a phosphate ester bond (NPL 1 and PTL 1 to 3).

On the other hand, in recent years, digitization has advanced in various fields, and analysis or the like using artificial intelligence (AI) has been performed. In such analysis, an analyzing subject has been shifted from human beings to AI, and required performance of a molecular probe has also been changed.

For example, for an AI capable of handling a large amount of data that cannot be processed by humans, the complexity of data is not a disadvantage, but an increase in the amount of data is an advantage. Furthermore, according to multiple regression analysis or the like by AI, analysis results of a plurality of molecular probes can be combined and handled.

### CITATION LIST

### Patent Literature

PTL 1: U.S. Pat. No. 6,479,650
PTL 2: U.S. Pat. No. 8,268,977

### PTL 3: Japanese Patent No. 6190534

### Non-Patent Literature

NPL 1: Lik Hang Yuen, et al., “Large-Scale Detection of Metals with a Small Set of Fluorescent DNA-Like Chemosensors”, Journal of the American Chemical Society 2014, Vol. 136, pp. 14576-14582

### SUMMARY

### The Problem

There has been a problem such that it is difficult to obtain sufficient data suitable for analysis or the like by AI with a conventional molecular probe. For example, the molecular probes as described in the above PTLs and NPL have a problem that they do not sufficiently interact with various types of target substances and it is difficult to sufficiently obtain detailed data acquisition.

Therefore, it is an object of the present invention to provide a composite luminescent signal generating material for state sensing which is more likely to interact with a target substance and can become a molecular probe capable of detecting, as a signal, a subtle change in emission color or emission spectral shape due to the interaction and acquiring a large amount of data converted from the signal in a short time and simply, as well as a carrier thereof, an ink, a measurement chip, and an analysis method using the same.

### Solution to Problem

In order to attain at least one of the aforementioned objects, a composite luminescent signal generating material reflecting one aspect of the present invention includes a nucleic acid structure and at least one luminescent compound residue bonded to a main chain of the nucleic acid structure, and emits, in response to a single excitation light, two or more types of luminescence selected from the group consisting of fluorescence, phosphorescence, excimer light emission, exciplex light emission, thermally activated delayed fluorescence, excited state intramolecular proton emission, triplet triplet annihilation emission, twisted intramolecular charge transfer emission, and aggregation-induced emission.

A luminescent substance carrier reflecting one aspect of the present invention includes: the composite luminescent signal generating material; and a carrier particle carrying the composite luminescent signal generating material.

A state sensing ink reflecting one aspect of the present invention includes: the composite luminescent signal generating material; and a solvent.

A state sensing method reflecting an aspect of the present invention includes: generating a signal by causing a target substance and the composite luminescent signal generating material to act on each other, and converting a state of the acting into an optical signal.

An analysis method reflecting one aspect of the present invention includes: disposing one of a target substance and the composite luminescent signal generating material into a reaction field configured to cause the target substance and the composite luminescent signal generating material to interact with each other, wherein the reaction field is in a plate; acquiring first signal information from the plate in which the target substance or the composite luminescent signal generating material is disposed; further disposing another one of the target substance and the composite luminescent signal generating material into the reaction field of the plate from which the first signal information has been acquired; acquiring second signal information from the plate in which the target substance and the composite luminescent signal generating material are disposed; and analyzing the first signal information and the second signal information by comparing the first signal information and the second signal information.

Another analysis method reflecting an aspect of the present invention includes: disposing one of a target substance and the luminescent substance carrier into a reaction field configured to cause the target substance and the composite luminescent signal generating material of the luminescent substance carrier to interact with each other, wherein the reaction field is in a plate; acquiring first signal information from the plate in which the target substance or the luminescent substance carrier is disposed; further disposing another one of the target substance and the luminescent substance carrier into the reaction field of the plate from which the first signal information has been acquired; acquiring second signal information from the plate in which the target substance and the luminescent substance carrier are disposed; analyzing the first signal information and the second signal information by comparing the first signal information and the second signal information.

Yet another analysis method reflecting an aspect of the present invention includes: putting one of a target substance and the composite luminescent signal generating material into a fluorescence intensity measurement cell; acquiring first signal information by using a fluorescence measurement device from the fluorescence intensity measurement cell in which the target substance or the composite luminescent signal generating material is put; disposing another one of the target substance and the composite luminescent signal generating material into the fluorescence intensity measurement cell from which the first signal information has been acquired; acquiring second signal information by using the fluorescence measurement device from the fluorescence intensity measurement cell in which the composite luminescent signal generating material and the target substance are disposed; and analyzing the first signal information and the second signal information by comparing the first signal information and the second signal information.

### Advantageous Effects of Invention

According to the composite luminescent signal generating material and the luminescent substance carrier according to an embodiment of the present invention, it is possible to easily interact with a target substance and it is possible to acquire a large amount of data. In addition, according to the analysis method according to one embodiment of the present invention, it is possible to analyze and target substances in detail using the above-described composite luminescent signal generating material.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram for explaining a composite luminescent signal generating material (luminescent dye molecule) according to one embodiment of the present invention.

FIG. 2 is a diagram for explaining the structure of a composite luminescent signal generating material (luminescent dye molecule) according to an embodiment of the present invention.

FIG. 3 is a diagram for explaining a method for producing a composite luminescent signal generating material (luminescent dye molecule) according to one embodiment of the present invention.

FIG. 4A-FIG. 4D are diagrams for explaining the mechanism by which a composite luminescent signal generating material (luminescent dye molecules) according to an embodiment of the present invention exhibits its effect.

FIG. 5 is a flowchart of an analysis method according to an embodiment of the present invention,

FIG. 6 illustrates the results of principal component analysis in Examples.

FIG. 7A illustrates linear discriminant analysis model plots when analyzing 7 types and 95 brands of beverages using the luminescent dye molecules 1 to 15, and FIG. 7B illustrates a confusion matrix at this time.

FIG. 8A illustrates linear discriminant analysis model plots when analyzing 7 types and 95 brands of beverages using the luminescent dye molecules 1 and 17 to 31, and FIG. 8B illustrates a confusion matrix at this time.

FIG. 9A illustrates linear discriminant analysis model plots when analyzing 7 types and 95 brands of beverages using the luminescent dye molecules 1 to 15 and 17 to 31, and FIG. 9B illustrates a confusion matrix at this time.

FIG. 10A illustrates linear discriminant analysis model plots when analyzing 7 types and 95 brands of beverages using the luminescent dye molecules 1 and 32 to 45, and FIG. 10B illustrates a confusion matrix at this time.

FIG. 11A illustrates linear discriminant analysis model plots when analyzing 7 types and 95 brands of beverages using the luminescent dye molecules 46 to 60, and FIG. 11B illustrates a confusion matrix at this time,

FIG. 12A illustrates linear discriminant analysis model plots when analyzing 7 types and 95 brands of beverages using the luminescent dye molecules 61 to 75, and FIG. 12B illustrates a confusion matrix at this time.

FIG. 13A illustrates linear discriminant analysis model plots when explanatory variables at the time of creating a discriminant model are replaced and 7 types and 95 brands of beverages are analyzed, and FIG. 13B illustrates a confusion matrix at this time.

### DETAILED DESCRIPTION OF EMBODIMENTS

Hereinafter, the present invention will be described in detail with reference to an embodiment. However, the present invention is not limited to these embodiments.

1. Composite Luminescent Signal Generating Material (Luminescent Dye Molecule) The composite luminescent signal generating material for sensing of the present embodiment (also referred to as “luminescent dye molecule” in the present specification) has a nucleic acid structure and at least one luminescent compound residue bonded to the main chain of the nucleic acid structure.

Note that in the present specification, the nucleic acid structure also includes a structure derived from one or more compounds selected from the group consisting of not only structures derived from DNA and RNA, but also, phosphorothioate oligodeoxynucleotide, 2′-O-(2-methoxy) ethyl-modified nucleic acid, siRNA, crosslinked nucleic acid, peptide nucleic acid, aTNA, SNA, GNA, LNA, and morpholino antisense nucleic acids.

Furthermore, the luminescent dye molecule may have only the portion including a luminescent compound residue (substituted with a nucleic acid base) bonded to the nucleic acid structure or the main chain of the nucleic acid structure (also referred to as “signal generation portion” in the present specification), or may have, in addition to the signal generation portion, various structures (also referred to as “base portion” in the present specification) linked to the signal generation portion. Note that the signal generation portion is a region which interacts with a target substance to generate a signal, and for example, it is sufficient that least one luminescent compound residue is attached to the main chain of the above-described nucleic acid structure, and the signal generation portion may include a region to which no luminescent compound residue is attached. The signal generation portion is preferably disposed on the tip side of the luminescent dye molecule, i.e., on the side that is more likely to come into contact with the target substance.

Here, for a single excitation light, the luminescent dye molecules of the present embodiment exhibit two or more types of luminescence selected from the group consisting of fluorescence, phosphorescence, excimer light emission, exciplex light emission, thermally activated delayed fluorescence, excited state intramolecular proton emission, triplet triplet annihilation emission, twisted intramolecular charge transfer emission, and aggregation-induced emission.

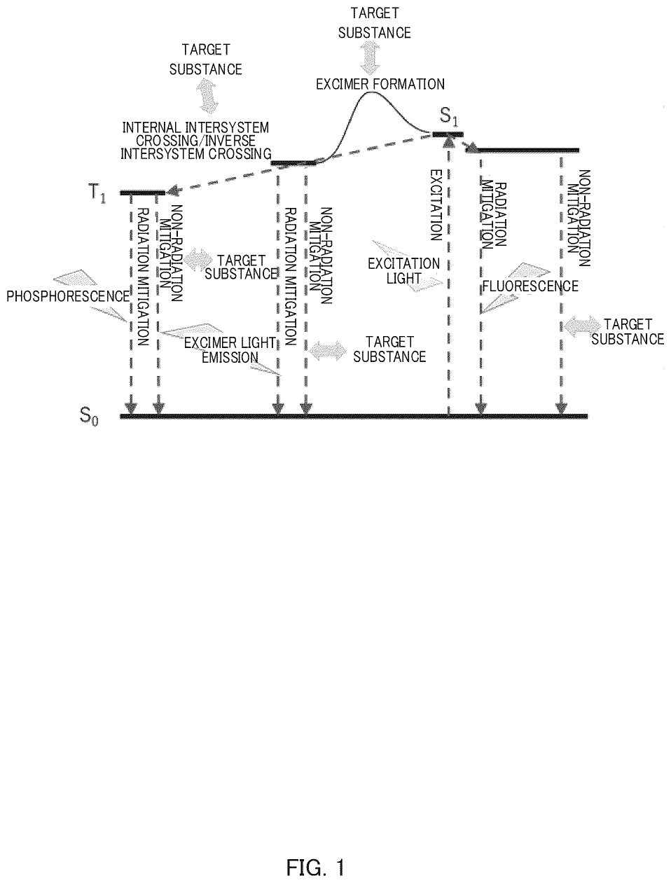

The luminescent dye molecule of the present embodiment can be used for analysis of the structure, state, and the like of a specific target substance. Specifically, when the luminescent dye molecule and the target substance are allowed to interact with each other, the structure and the electronic state of a luminescent compound residue (also referred to as a “chromophore” or a “luminophore” in the present specification) in the luminescent dye molecule change, and complicated light emission behavior different from the case of the luminescent dye molecule alone is obtained. For example, as illustrated in FIG. 1, when a luminescent dye molecule that emits three different types of light, i.e., fluorescence, phosphorescence, and excimer light emission, in response to a single excitation light is allowed to interact with a target substance, the processes of fluorescence, phosphorescence, and excimer light emission are changed by the interaction between the target substance and the luminescent dye molecule, and the wavelengths and lifetimes of the light are changed. Therefore, a large amount of complicated data in which these lights are combined is obtained according to the structure, the state, and the like of the target substance. According to such a complicated and large amount of data, it is possible to grasp the structure, the state, and the like of the target substance in very detail.

Examples of the structure having a nucleic acid structure and at least two luminescent compound residues bound to the main chain of the nucleic acid structure include a structure (or molecule) having a main chain having at least one structural unit including a sugar structure derived from a pentose or hexose and a phosphate ester bond bonded to the sugar structure, and one or more chromophores or luminophores bound to the sugar structure.

In this case, it is preferable that 50% or more of the sugar structure to which a chromophore or a luminophore is bonded is in a β-form in the signal generation portion of the luminescent dye molecules of the present embodiment. In general, DNA has a structure in which bases are bound to a main chain (deoxyribose) including phosphate ester bonds and a deoxyribose-derived structure, but in natural DNA, all the deoxyribose in the main chain is in the β-form. Therefore, when 50% or more of the sugar structure to which or a chromophore or a luminophore is bonded is a β-form, it can be said that the saccharide has high structural similarity to substances (target substances) existing in nature, such as DNA and RNA. When such a luminescent dye molecule is mixed with a target substance, steric hindrance or the like is less likely to occur, and the luminescent dye molecule can enter the inside or follow the shape of the target substance. Therefore, the target substance can be analyzed in more detail.

In addition, in the signal generation portion of the luminescent dye molecule of the present embodiment, more preferably, 80% or more of sugar structures to which a chromophore or a luminophore is bonded are β-form, and still more preferably, all of them are β-form. Whether the sugar structure having the chromophore or the luminophore bonded thereto is a β-form or an α-form can be confirmed by NMR analysis, X-ray crystal structure analysis, or the like.

Hereinafter, the specific structure of the signal generation portion of the luminescent dye molecule will be described.

The main chain of the signal generation portion of the luminescent dye molecule having a sugar structure derived from pentose or hexose may at least one structural unit including a sugar structure derived from pentose or hexose and a phosphate ester bond bonded to the sugar structure. The main chain may include only one of the structural units, or may include a plurality of the structural units. That is, the main chain may have a structure having one of the above sugar structure and one phosphate ester bond bonded to the sugar structure, or a structure alternately containing the sugar structure and phosphate ester bond. Usually, both ends of the main chain of the luminescent dye molecule have a sugar structure, so that the number of sugar structures is larger than the number of phosphate ester bonds by one. When the main chain includes a plurality of structural units, the plurality of structural units may be the same as or different from each other.

The number of the structural units contained in the main chain of the signal generation portion of the luminescent dye molecule is appropriately selected depending on the type of the target substance or the like, and is preferably 2 or more and 6 or less. When the amount of the structural unit is increased, the luminescent dye molecule tends to act specifically on the target substance. However, in the present embodiment, it is preferable to obtain a large amount of data by allowing the luminescent dye molecules to interact with various positions of the target substance. Therefore, it is preferable that the luminescent dye molecule and the target substance have moderate (not excessive) specificity, and the number of the structural units is preferably 6 or less.

The main chain of the signal generation portion of the luminescent dye molecule may partially contain a structure other than the sugar structure derived from pentose or hexose and the structural unit containing a phosphate ester bond as long as the object and effect of the present embodiment are not impaired. The structure of both ends of the main chain is not particularly limited and may be various structures such as an OH group and an alkoxy group.

Here, examples of the pentose include ribose, deoxyribose, and xylose. On the other hand, specific examples of the hexose include allose, glucose, mannose and the like. Among these, the sugar structure is particularly preferably a structure derived from ribose or deoxyribose, since the main chain of the luminescent dye molecule has the same structure as the main chain of DNA or RNA and thus tends to interact with DNA or RNA.

When the structural unit contains a structure derived from ribose or deoxyribose, the phosphate ester bond is preferably bonded to the carbon at position 3 and the carbon at position 5 of ribose or deoxyribose. Furthermore, the below-described chromophore or luminophore is preferably bonded to the 1-position of ribose or deoxyribose. That is, the luminescent dye molecules of the present embodiment preferably include a structure represented by the following general formula (1a) or (1b).

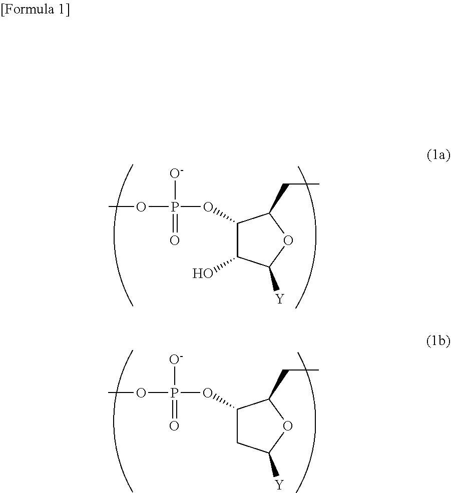

In the general formulae (1a) and (1b), Y represents a chromophore or a luminophore which will be described later.

On the other hand, as described above, the structural unit constituting the main chain of the nucleic acid structure of the signal generation portion is not necessarily limited to a structural unit containing a sugar structure derived from pentose or hexose and a phosphate bond, such as DNA or RNA. Typical examples of the other structural unit include a peptide nucleic acid-type structural unit as described below.

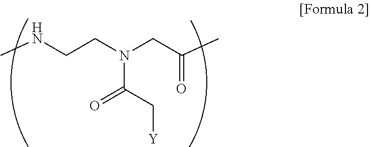

in a similar manner as in DNA and/or RNA, peptide nucleic acids can be synthesized exhaustively by using a commercially available automatic synthesizer (peptide synthesizer). Since the peptide nucleic acid has no electric charge and no electrostatic repulsion, it can form a stronger association with the target substance. Furthermore, since it is resistant to enzymes such as nuclease and protease, it can be used for cells. Furthermore, relatively large-scale synthesis is possible. Provided that due to the nonionic structure, it aggregates in water and the solubility decreases in some cases. Therefore, when the signal generation portion has a structure derived from a peptide nucleic acid, it is preferable to appropriately select the type of the base portion to be linked to the signal generation portion or to select the solvent.

Note that in the following description, a case where the structural units constituting the main chain include a sugar structure and a phosphate ester bond will be described as an example.

The luminescent compound residue (chromophore or luminophore) contained in the signal generation portion of the luminescent dye molecule may have any structure that emits a predetermined type of light alone in response to a single excitation light, or emits a predetermined light by the action of a plurality of chromophores or luminophores. The chromophore or the luminophore is preferably bonded to the sugar structure of the main chain so that the sugar structure is a β-form. Note that in the present specification, the term “chromophore” refers to a structure that absorbs light having one or more wavelengths, and the term “luminophore” refers to a structure that absorbs light having one or more wavelengths to emit light.

The number of the chromophore or the luminophore included in the signal generation portion of the luminescent dye molecule may be only one, provided that the luminescent dye molecule can exhibit a plurality of types of luminescence. However, from the viewpoint that the luminescent dye molecule is more likely to exhibit a plurality of types of luminescence, the number is preferably 2 or more, and more preferably 3 or more and 6 or less. When the signal generation portion of the luminescent dye molecule has a plurality of chromophores or luminophores, the number of types thereof may be only one, or may be two or more. Here, in the luminescent dye molecule, usually, one chromophore or one luminophore is bonded to one sugar structure in the main chain. Therefore, when the luminescent dye molecule has two or more chromophores or luminophores, the sugar structure in the main chain is also preferably two or more. That is, the number of chromophores or lumiphores in the luminescent dye molecule is preferably equal to or smaller than the number of sugar structures (or peptide structures) in the main chain of the signal generation portion.

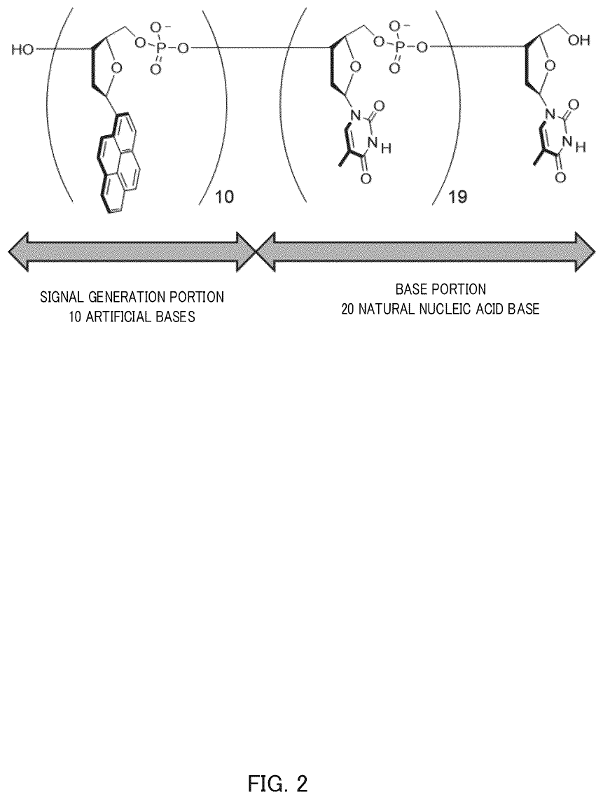

Note that when the number of chromophores or luminophores of the signal generation portion in the luminescent dye molecule is smaller than the number of sugar structures (or peptide structures) in the main chain, some of the sugar structures are in a state where no chromophore or luminophore is bonded thereto. The sugar structure to which a chromophore or a luminophore is not bonded may not have another atomic group or the like bonded thereto but may have a natural nucleic acid base bonded thereto as long as the object and effect of the present embodiment are not impaired. As used herein, the natural nucleic acid base refer to adenine, guanine, cytosine, thymine, or uracil. However, the total number of the natural nucleic acid bases bonded to the main chain is preferably 50% or less, and more preferably 25% or less, based on the total number of the structural units constituting the main chain structure of the region (signal generation portion) mainly responsible for the interaction in the luminescent dye molecule. The luminescent dye molecule illustrated in FIG. 2, which has a signal generation portion (a portion interacting with a target substance) and a base portion (a portion hardly contributing to the interaction), and in which the signal generation portion is composed of the 10th sugar chain from the tip, will be described as an example. In this case, the number of natural nucleic acid bases bound to the sugar chain is preferably 50% or less, and more preferably 25% or less, based on the total number of sugar structures contained in the sugar chain, in the sugar chains from the first to the 10th (signal generation portion). When the number of the natural nucleic acid bases in the signal generation portion is 50% or less, the association between the luminescent dye molecules is suppressed, and the interaction between the target substance and the luminescent dye molecules tends to be dominant. In a case where the target substance has a large molecular size, such as a protein or a liposome, the signal generation portion may continue beyond the sugar chains from the first to the 10th, and in this case, those having a structure in which a large number of natural bases are linked to the 10th and subsequent sugar chains from the tip are also preferable from the viewpoint of the overall affinity. In this case, the number of natural bases to be bound to the signal generation portion is preferably about 10 to 50 bases, and more preferably 15 to 30 bases. Further, in the signal generation portion, the natural nucleic acid base and the non-natural nucleic acid base are preferably bonded such that the sugar structure is a β-form as described above. Note that the structure of the base portion is not limited to the above, and various structures may be employed.

Here, examples of the chromophore or the luminophore that emits fluorescence include structures derived from fluorescein, rhodamine, boron dipyrromethene, and the like. Examples of the chromophore or luminophore that emits phosphorescence include structures derived from iridium complexes, platinum complexes, and the like. Examples of the chromophore or luminophore that emits excimer light include structures derived from pyrene, anthracene, perylene, and the like. Examples of the chromophore or the luminophore that emits exciplex light include a structure derived from pyrene-dimethylaniline or the like. Examples of the chromophore or the luminophore that emits thermally activated delayed fluorescence include structures derived from 4CzIPN, DABNA, and the like. Examples of chromophores or luminophores which emit excited state intramolecular proton emission include structures derived from hydroxyphenylbenzoxazole and the like. Examples of chromophores or luminophores that emit triplet triplet annihilation emission include structures derived from 9,10-diphenylanthracene, rubrene, and the like. Examples of chromophores or luminophores that emit twisted intramolecular charge transfer emission include structures derived from diaminoanthracene, diaminonaphthalene, and the like. Examples of chromophores or luminophores which emit aggregated organic luminescence include structures derived from tetraphenylethene, hexaphenylsilole and the like.

Furthermore, in the embodiment, a luminescent compound used as a light emitting material or host, an electron transport material, a hole transport material, or a light emitting material of an organic EL can also be suitably used as the chromophore or the luminophore. Specific examples of such a luminescent compound include compounds described in “Leading-edge Organic EL” (CMC Publishing Co., Ltd), Organic EL Material Technology (CMC Publishing Co., Ltd), all of Organic EL (Japan Practical Industrial Publishing Co., Ltd), various dye materials (Kagaku Dojin Co., Ltd), and the like.

The reasons why various compounds for organic EL are suitable as the chromophore or the luminophore of the signal generation portion of the luminescent dye molecule of the present embodiment are as follows. For example, the electron transport material is a substance containing an electron-accepting aromatic compound that tends to become an anion radical, and therefore strongly interacts with an electron-rich compound in the target substance. Conversely, the hole transport material is a substance containing an electron-donating aromatic compound that easily becomes a cation radical, and thus strongly interacts with an electron-deficient compound in the target substance. Since a light emitting material of an organic EL has both of the properties and is a substance having a high emission quantum yield, a strong emission signal is obtained. Note that a phosphorescent material or a delayed fluorescent material can also be used. These materials are preferred as the materials emit light with a time delay of about nanoseconds to microseconds relative to conventional fluorescence emission, and thus can include a time factor in the number of dimensions as a sensing material and can be multi-dimensionalized as data for use in machine learning, deep learning, or the like.

A typical example of the organic EL material is described below. These are molecular groups that can be the Y in the above-described general formulae (1a) and (1b). Furthermore, it may become Y via a linking group or the like.

### Electron Transport Material

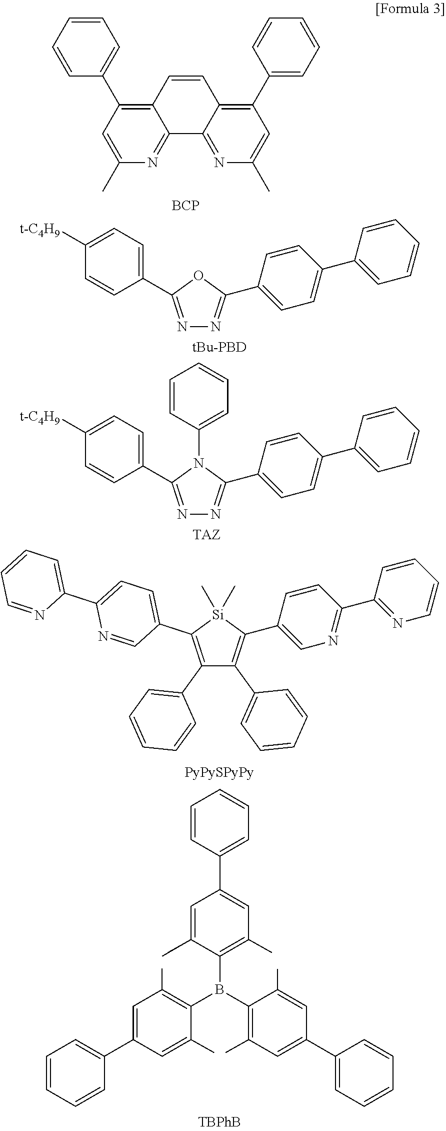

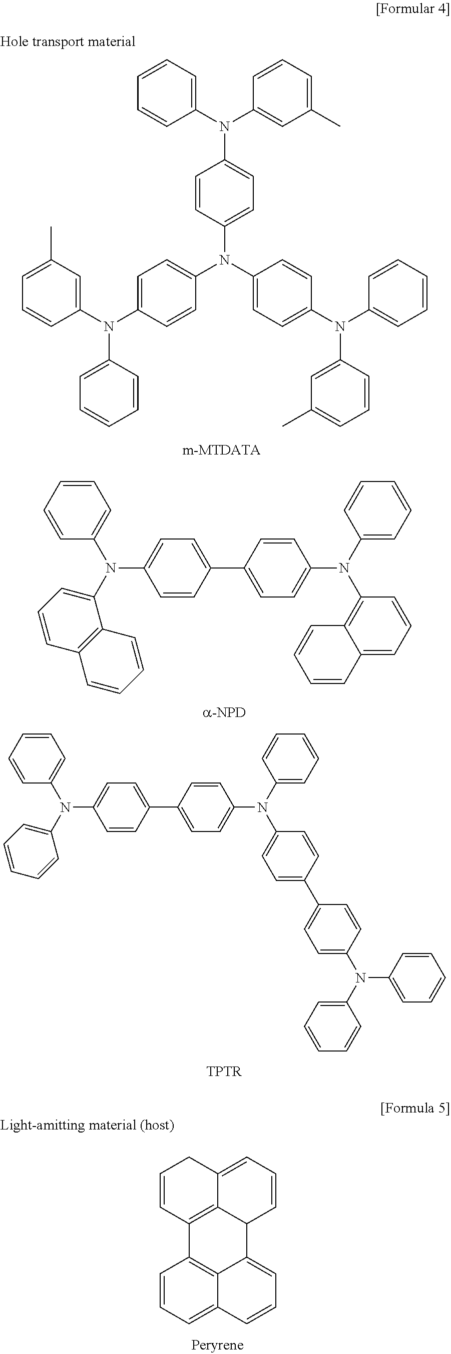

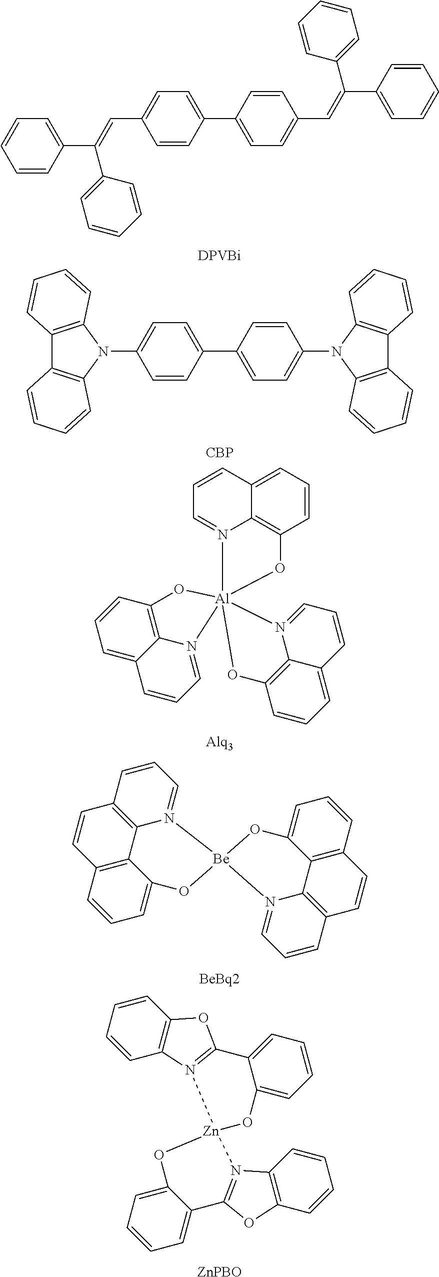

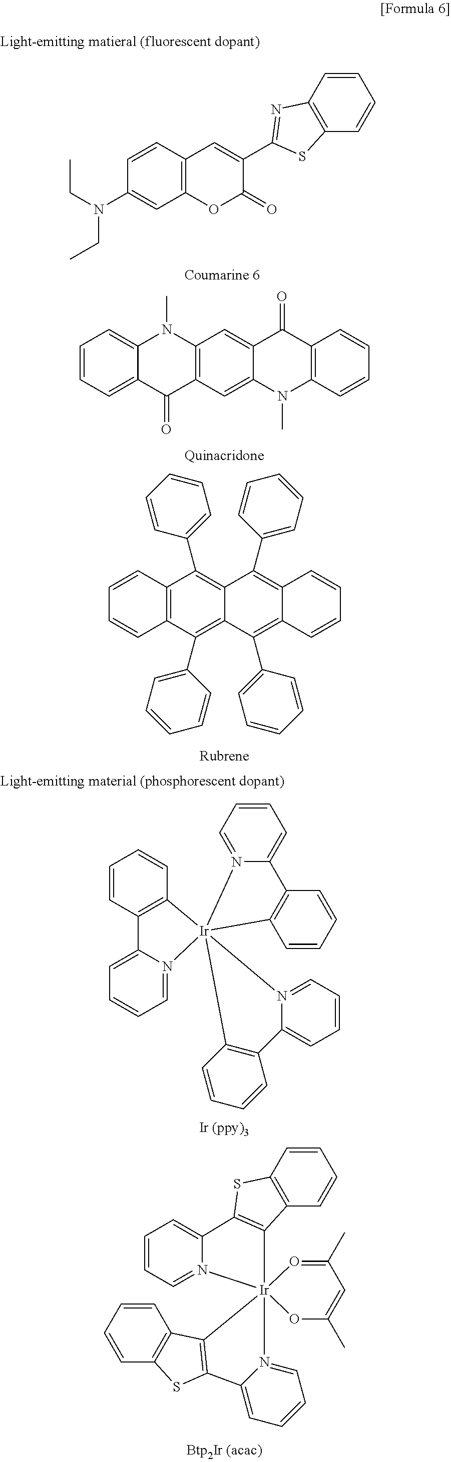

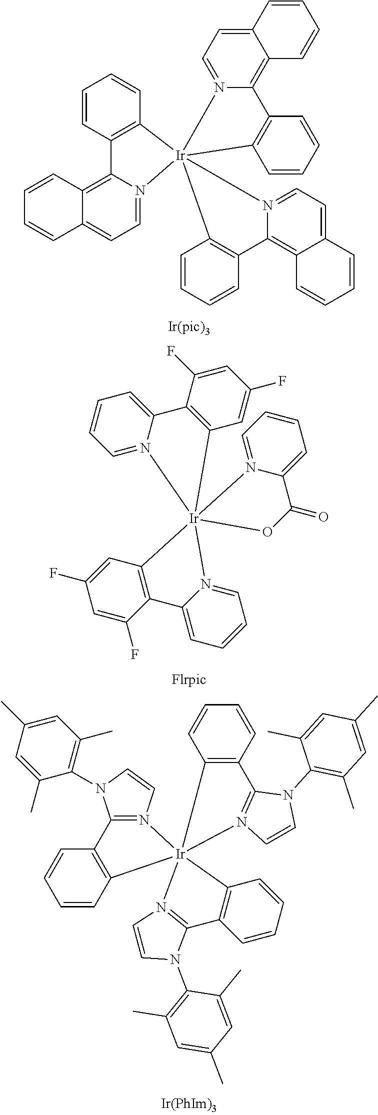

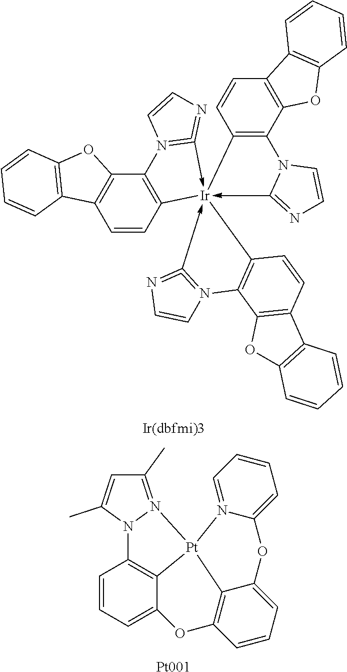

Examples of chromophores or luminophores other than those described above include the following.

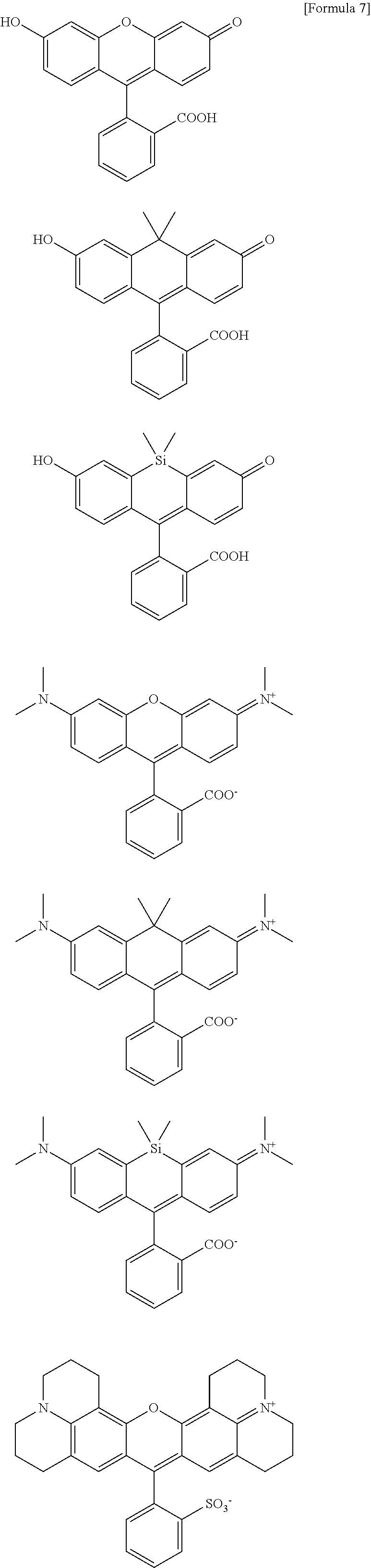

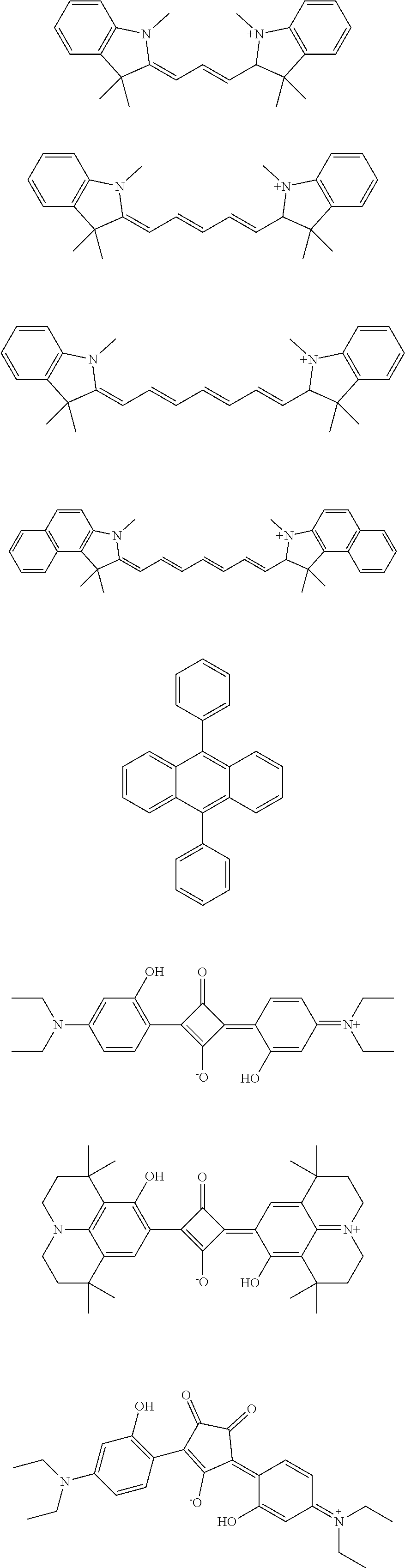

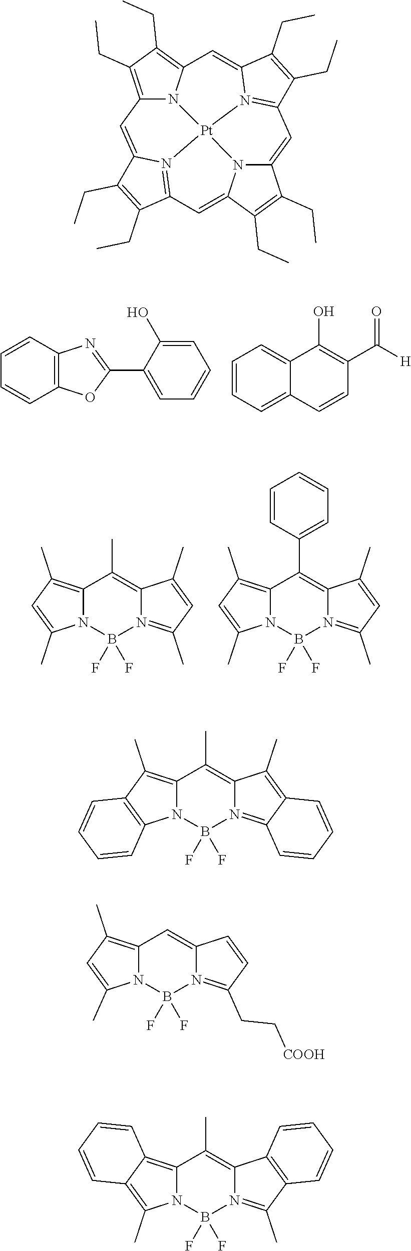

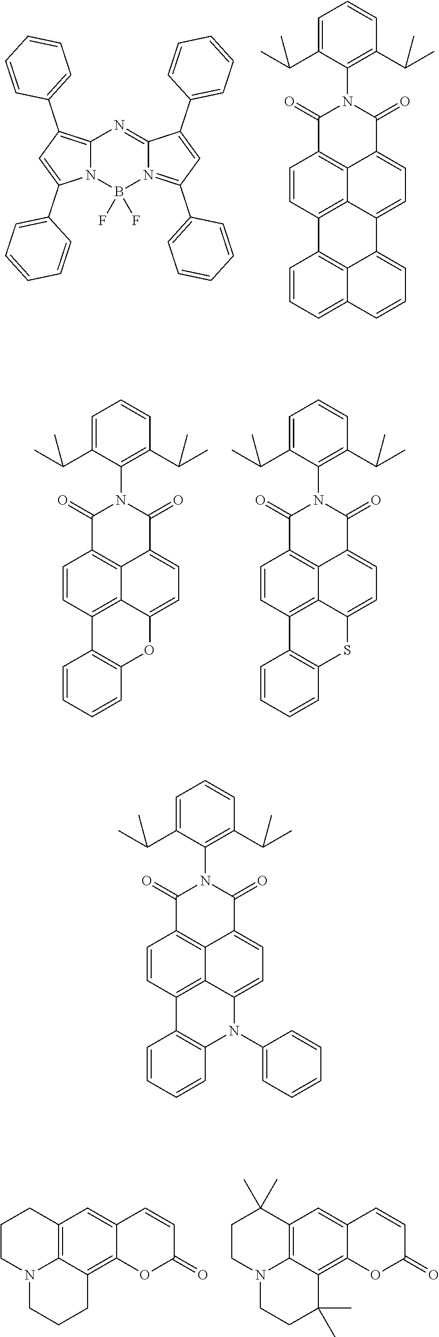

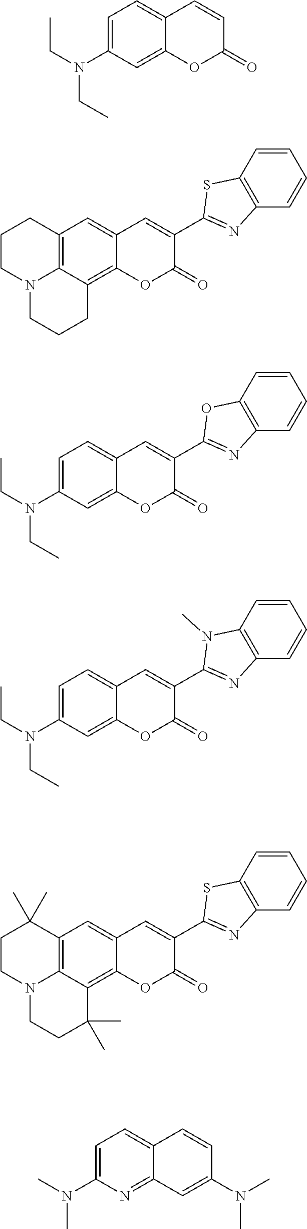

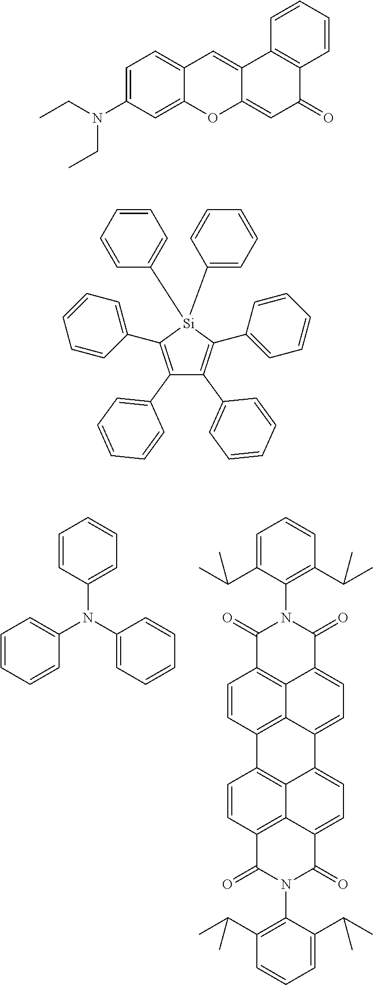 [SMILES: CCN(CC)c1ccc2cc3c4ccccc4c(=O)cc-3oc2c1 ; c1ccc(C2=C(c3ccccc3)[Si](c3ccccc3)(c3ccccc3)C(c3ccccc3)=C2c2ccccc2)cc1 ; c1ccc(N(c2ccccc2)c2ccccc2)cc1 ; CC(C)c1cccc(C(C)C)c1N1C(=O)c2ccc3c4ccc5c6c(ccc(c7ccc(c2c37)C1=O)c64)C(=O)N(c1c(C(C)C)cccc1C(C)C)C5=O]

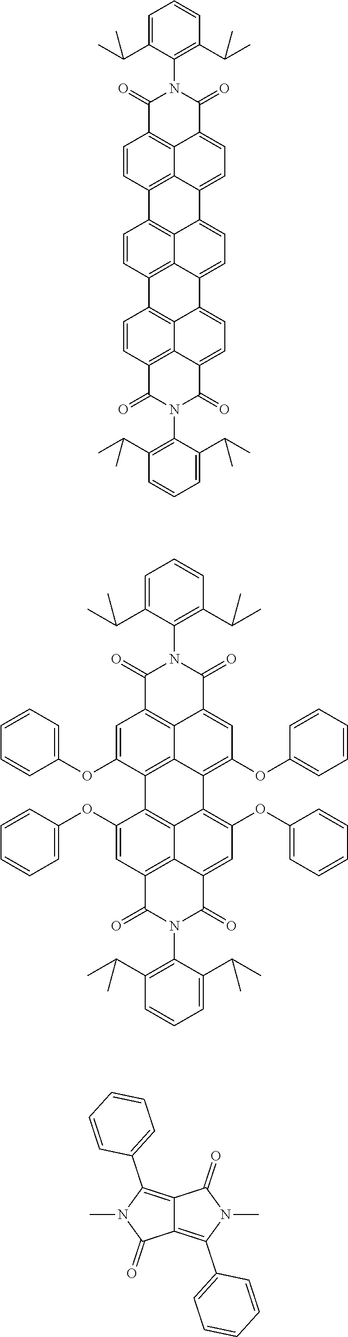 [SMILES: CC(C)c1cccc(C(C)C)c1N1C(=O)c2ccc3c4ccc5c6ccc7c8c(ccc(c9ccc(c%10ccc(c2c3%10)C1=O)c4c59)c86)C(=O)N(c1c(C(C)C)cccc1C(C)C)C7=O ; CC(C)c1cccc(C(C)C)c1N1C(=O)c2cc(Oc3ccccc3)c3c4c(Oc5ccccc5)cc5c6c(cc(Oc7ccccc7)c(c7c(Oc8ccccc8)cc(c2c37)C1=O)c64)C(=O)N(c1c(C(C)C)cccc1C(C)C)C5=O ; CN1C(=O)C2=C(c3ccccc3)N(C)C(=O)C2=C1c1ccccc1]

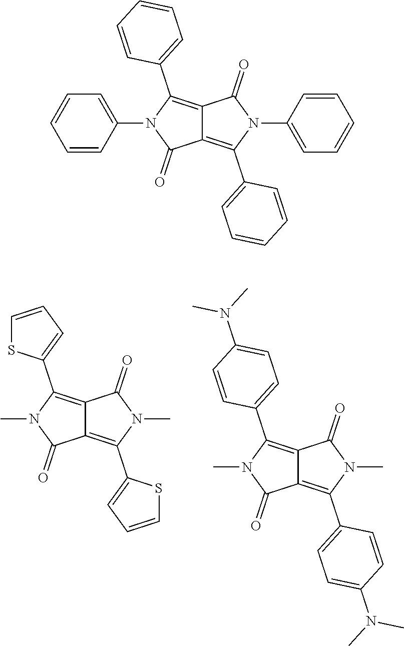

Furthermore, in the present embodiment, a non-luminescent monomer may also include, as a moiety for controlling the interaction between the luminescent dye molecule and the target substance, a structure having various functions in the luminescent dye molecule. For example, structures having the following functions can be allowed to coexist in the molecular chain of the luminescent dye molecule. Representative functions and examples of monomer structures for realizing the functions are shown below.

### A1) Compound Capturing Metal Ion

Typical examples thereof include those having a ligand structure forming a chelate. Examples of a form of the coordination includes N, N coordination, N, O coordination, O, O coordination, N, S coordination, O, S coordination, S, S coordination, N, Se coordination, O, Se coordination, S, Se coordination, Se, Se coordination and the like. Specific examples include 2,2′-bipyridine, phenanthrolines, 1,8-diaminonaphthalene, amino acids, cryptands, crown ethers, 8-hydroxyquinoline, 3-mercaptopropanol, 3-mercaptopropionic acids, thiocatechol, salicylaldehyde, acetoacetic esters, β-diketones and the like.

### A2) Compound Whose Molecular Shape Changes Depending on Wavelengths of Light to be Emitted

Typical examples thereof include azobenzene, stilbene, fulgide, diarylethene, spiropyran, spirooxazine, dihydropyrene, phenoxyquinone, biindenylidene, cobalt complex, and imidazole dimer.

### A3) Compounds Acting as Lewis-Acid

Typical examples include Group 13 elements and transition metals, and specific examples include triarylborane, trialkylborane, trialkoxyborane, trifluoroborane, trichloroborane, and tribromoborane.

### A4) Compounds Acting as Lewis-Base

Typical examples include Group 15, 16 elements and the like, and specific examples include arylamine, alkylamine, arylphosphine, alkylphosphine, arylether, alkylether, arylsulfide, alkylsulfide and the like.

A5) Compound Interacting with Peptide or Protein

Typical examples include nucleic acid base, transition metal complex, oxo acid, and the like, and specific examples include adenine, thymine, guanine, thymine, cytosine, a zinc complex, a copper complex, a nickel complex, a cobalt complex, tungstic acid, molybdic acid, phosphoric acid, and the like.

### A6) Compound Forming Hydrophobic-Hydrophobic Interaction

Typical examples include straight chain alkanes, straight chain alkenes, straight chain alkynes, branched alkanes, branched alkenes, branched alkynes, aromatic rings, heteroaromatic rings and the like.

### A7) Compound Forming Dipole-Dipole or Quadrupole-Quadrupole Interaction

Typical examples include alkyl halides, aryl halides, nitriles, nitroarenes, anilines, anisoles, heterocyclic compounds, and the like.

In the present embodiment, the luminescent dye molecule preferably contains, as a chromophore or a luminophore, at least one structure selected from a structure emitting fluorescence, a structure emitting excimer light emission, and a structure emitting exciplex light emission, and preferably contains at least a structure emitting fluorescence. In a case where the luminescent dye molecule emits fluorescence, there is an advantage that it is easy to analyze with various measurement devices.

Furthermore, it is preferable that the luminescent dye molecules exhibit a plurality of types of luminescence by irradiation with light having wavelengths of 300 to 400 nm. When the luminescent dye molecule exhibits a plurality of types of luminescence by irradiation with light having the wavelength, a special light source is not required and the target substance is less likely to be damaged when the target substance is analyzed.

Provided that when LEDs or organic EL elements are used as the excitation light sources, it is advantageous to excite in the visible light region, and therefore, in such a case, 400 to 700 nm is effective as the absorbing wavelengths of the aforementioned luminescent dye molecules, and such a dye can also be used.

The molecular weight of the luminescent dye molecule (the molecular weight of the signal generation portion in the case of having the signal generation portion and the base portion) is appropriately selected depending on the type of the chromophore or the luminophore of the luminescent dye molecule, the length of the main chain and the like, but is usually preferably 500 or more and 10000 or less, more preferably 500 or more and 4000 or less. When the molecular weight of the luminescent dye molecule is 10,000 or less, the specificity for the target substance becomes moderately low, and it becomes possible to cause the luminescent dye molecule to nonspecifically react with a plurality of sites of the target substance.

### (Method for Producing Luminescent Dye Molecule)

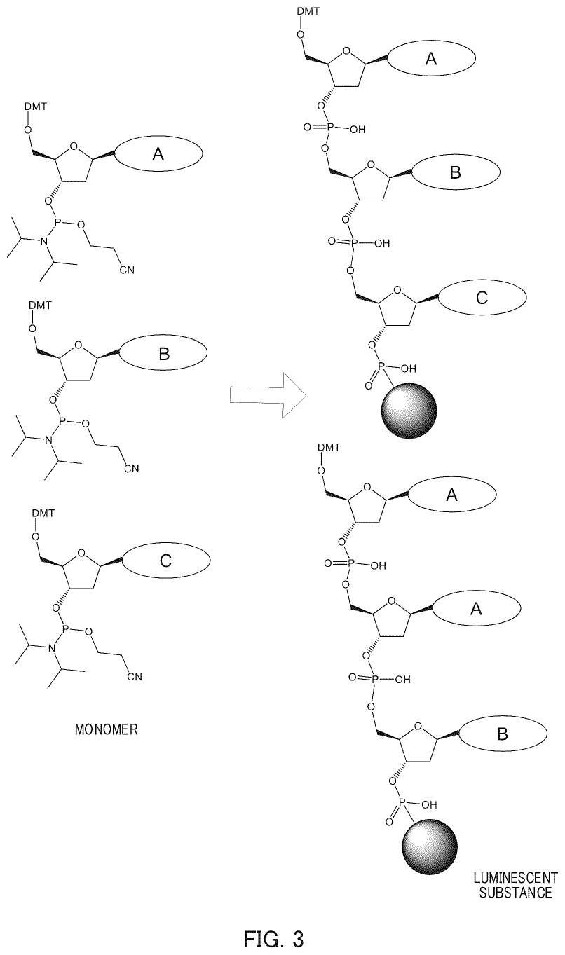

The method for producing the luminescent dye molecule is appropriately selected depending on the type of the nucleic acid structure in the luminescent dye molecule. For example, the luminescent dye molecule having the above-described sugar structure can be produced by the following method. A monomer in which the chromophore or the luminophore and the phosphate ester are bonded to pentose or hexose is prepared. It can be synthesized by polymerizing the above-mentioned monomers in a desired sequence using a phosphoramidite method with a DNA/RNA synthesizer or the like. According to such a method, for example, as illustrated in the schematic view of FIG. 3, a plurality of types of monomers (three types in FIG. 3) having different types of chromophores or luminophores (represented by A, B, and C in FIG. 3) are prepared, and a desired number of the monomers can be bonded by changing the arrangement order of the monomers (three monomers can be bonded in FIG. 3). That is, a wide variety of luminescent dye molecules can be synthesized from a plurality of types of monomers having different types of chromophores or luminophores. In the example illustrated in FIG. 3, 27 type of luminescent dye molecule can be synthesized. By changing the type of the monomer to be used and the number of bonds of the monomer, a very large number of types of luminescent dye molecules can be synthesized.

Note that when the phosphoramidite method is performed, usually, a part of monomers, for example, a hydroxyl group of a sugar structure (e.g., a hydroxyl group bonded to the carbon at position 3 of ribose or deoxyribose) or a hydroxyl group derived from phosphoric acid is supported on a particulate carrier (herein also referred to as “carrier particle”) to perform a polymerization reaction. The carrier particles are, for example, porous glass, porous silica gel, polystyrene, or the like, and the porous glass includes a porous body of a metal oxide such as silica gel or alumina. After the synthesis of the luminescent dye molecules (polymerization of the monomers), the carrier may be removed to obtain only the luminescent dye molecules, or the luminescent dye molecules may be used in a state of being carried on carrier particles (also referred to as a “luminescent substance carrier” in the present specification). Furthermore, the luminescent dye molecules and a solvent may be mixed (the luminescent dye molecules may be dispersed in the solvent) to form ink (referred to as “ink for state sensing” in the present specification). Further, they may be used as a measurement chip in which they are immobilized linearly, two dimensionally, or three dimensionally.

### (Effect of Luminescent Dye Molecule)

As described above, a representative luminescent dye molecule of the present embodiment has a main chain structure similar to that of a substance existing in the natural world (for example, DNA or RNA). Therefore, it is possible to easily interact with various target substances, and according to the luminescent dye molecule, it is possible to grasp the state of the target substance in detail. In addition, the above-described luminescent dye molecules exhibit a plurality of types of luminescence by irradiation with light having a specific wavelength. Therefore, it is possible to obtain a large amount of complicated emission data according to the state of the target substance, and it is possible to analyze the target substance in very detail.

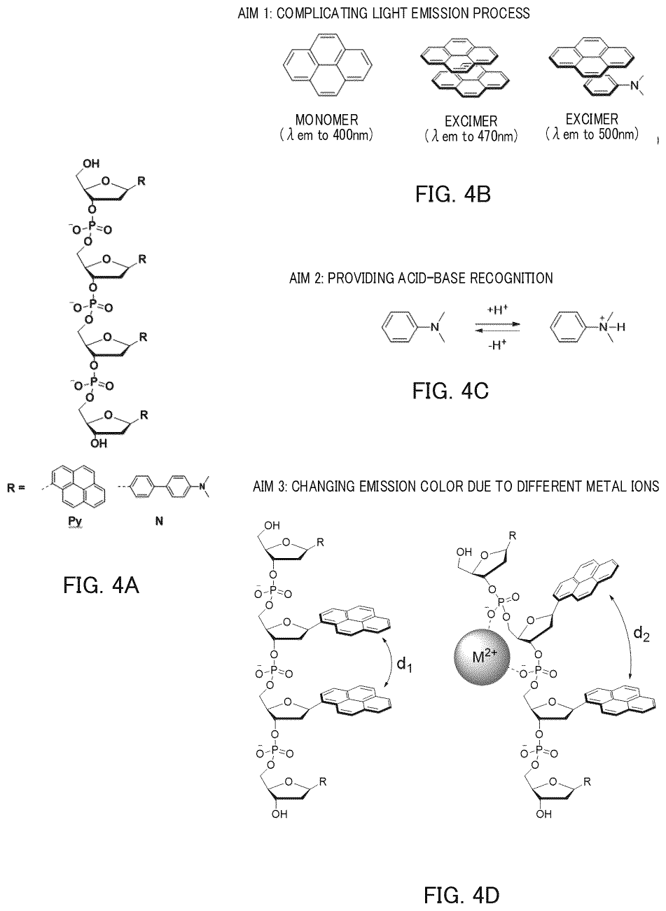

The mechanism of exhibiting the effect is schematically illustrated in FIGS. 4A to 4D. First, for the purpose of promoting understanding, the luminescent dye molecule of the present embodiment is defined as a molecule having four sugar structures and four luminophores as illustrated in FIG. 4A. All of the four R's may be pyrene (Py in FIG. 4), dimethylaminobiphenyl (N in FIG. 4), or a mixture thereof. One or two of the four R's may be a hydrogen atom. With such a molecular structure, a form of a complex-type luminescent dye molecule can be constructed.

This embodiment is characterized in that a liquid substance, a dispersion substance, or a gaseous substance as a target substance causes a complex interaction with the luminescent dye molecule of the present invention or the carrier thereof, and a large amount of multidimensional data is generated as a light or color signal. FIG. 4B to FIG. 4D show the concept most simply and concretely, and the aim is roughly divided into three cases.

For example, assuming that all of the above-mentioned R's are pyrene, when a specimen is brought into contact with a liquid or a medium in which the luminescent dye molecule is present and excited with ultraviolet light, and when a target substance (e.g., an aliphatic compound or the like) which is contained in the specimen, is inserted between pyrene molecules present in the luminescent dye molecule, and has a wide band gap that does not quench fluorescence of pyrene is present, monomer emission of pyrene illustrated in the left drawing of FIG. 4B is observed from the luminescent dye molecule.

When no component that interacts with pyrene is contained in the specimen, excimer light emission of pyrene is obtained as illustrated in the center diagram of FIG. 4B. Furthermore, when a target substance (fluorescent substance) having an energy level close to the band gap of pyrene that interacts with pyrene is present in the specimen, exciplex light emission due to pyrene and the target substance (the fluorescent substance) is obtained, as in the right diagram of FIG. 4B.

Furthermore, in a case where a target substance (metal ion) such as a sodium ion or a calcium ion exists in the specimen, the target substance forms a chelate with a phosphate group existing in the main chain part of the luminescent dye molecule, but as illustrated in FIG. 4D, the intermolecular distance between pyrene and pyrene changes according to the size of the metal ion, and therefore, the emission color (emission spectrum) of the excimer light emission itself also changes.

In addition, in a case where R in the structure illustrated in FIG. 4A is dimethylaminobiphenyl (N), a change in the emission color (spectrum) of the fluorescent dye (N) occurs due to the proximity of an acid and a base as illustrated in FIG. 4C. This is different from acid-base ion pairing, such as switching on/off like mineral acids (such as sulfuric acid and nitric acid) and alkali metals. In this case, the distance of approach to N continuously changes depending on the acidity (ease of donation of protons) of a substance contained on the analyte side. Therefore, using this light emission phenomenon as a signal leads to expansion of the dynamic range. Since N is a Lewis base, the same interaction occurs with Lewis acidic substances (for example, triarylborane, trialkylaluminum, tetraalkoxytitanium, and the like) as well as protic acidic substances. Therefore, even when such a target substance is contained in the specimen, a specific emission color change occurs.

Next, in the case where R of the luminescent dye molecule is alternately pyrene (Py) and dimethylaminobiphenyl (N), unlike the above case, when there is no interaction between the specimen and pyrene or dimethylaminobiphenyl, the exciplex light emission of pyrene and dimethylaminobiphenyl is observed. On the other hand, in the case of interaction, similarly to the above, complicated luminescence due to a mixed exciplex of the analyte component and pyrene and/or dimethylaminobiphenyl is obtained.

When one or two of the four R's on the inner side are hydrogen atoms, excimer light emission or exciplex light emission does not occur from the luminescent dye molecule itself, or the contribution thereof is small, and almost monomer emission is observed, but since the steric hindrance of the hydrogen atoms is small, the interaction between the target substance in the specimen and R of the luminophore is enhanced, and the change in the luminescent signal is enhanced.

When Py or N in FIG. 4A is not a normal fluorescent substance but a phosphorescent compound or a thermally excited delayed fluorescent compound, light emission occurs at a timing delayed by several tens of nanoseconds to several microseconds from immediately after excitation. Therefore, the factor of “time” rather than the emission color expands the dynamic range, and such a phenomenon can also be applied to the present invention in addition to the mechanism as illustrated in FIGS. 4B to FIG. 4D.

Such an intermolecular interaction and a resultant subtle change in emission color or emission spectrum, a delay of emission of several microseconds, or the like, and furthermore, an extremely subtle change in a light emission phenomenon brought about by metal chelate formation due to a main chain structure have not been able to be applied as analysis information for human understanding. However, on the premise that artificial intelligence (AI) that has become generally available in recent years and machine learning and informatics utilizing the AI are used, such a wide variety of light emission phenomena beyond the understanding of human beings are state description data corresponding to a target specimen. Such a new concept is a fundamental concept of the present invention and is extremely useful as a new method of state description for various future research and development and production processes, and furthermore, for complicated samples such as cell culture and waste liquid/waste or water/sludge treatment.

Examples of analysis using DNA-like fluorescent compounds having similar structures (NPL 1, PTLs 1 to 3, etc) are disclosed. However, they are largely different from the concept of the present invention of multidimensionally measuring a complex system as it is and inductively obtaining a solution using AI as described above, and thus are considered to be completely different inventions.

### 2. Analysis Method

Hereinafter, an example of an analysis method using the luminescent dye molecule including the signal generation portion will be described, but the analysis method using the luminescent dye molecule is not limited to the method.

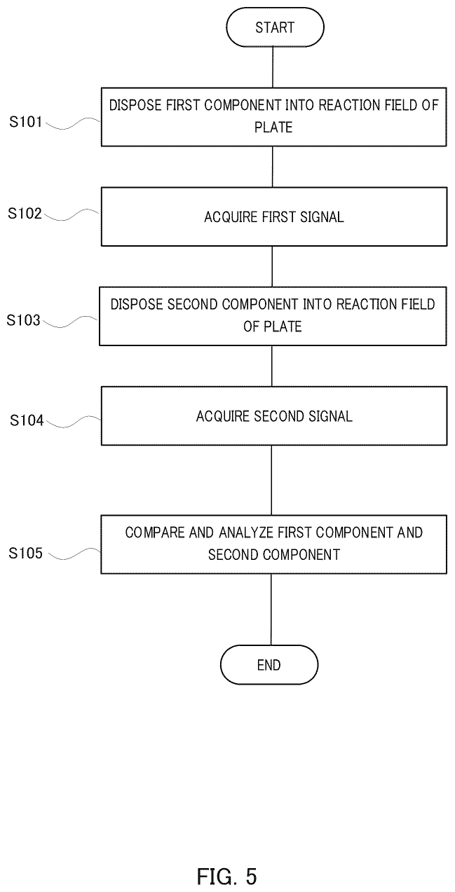

A flow chart of the analysis method is illustrated in FIG. 5. In the analysis method, one (hereinafter, also referred to as a “first component”) of the luminescent dye molecule and the target substance is disposed in a reaction field of a plate including the reaction field for allowing the luminescent dye molecule and the target substance to interact with each other (S101, hereinafter, also referred to as a “first component disposing step”). Next, first signal information is acquired from the plate in which the first component is disposed (S102, also referred to as “first signal information acquiring step”). Then, the other one (hereinafter also referred to as “second components”) of the luminescent dye molecule and the target substances is further disposed in the reaction field of the plate from which the first signal information has been acquired (S103, hereinafter also referred to as “second components disposing step”). Then, second signal information is acquired from the plate in which the luminescent dye molecule and the target substance are disposed (S104, hereinafter also referred to as a “second signal acquisition step”). Thereafter, the first signal information and the second signal information are compared and analyzed by an analysis unit (S105, hereinafter also referred to as an “analyzing step”). Thereafter, the first signal information and the second signal information are compared and analyzed (S105, hereinafter also referred to as an “analyzing step”).

The type of the target substance to be analyzed by the analysis method of the present embodiment is not particularly limited, and for example, the target substance may be a substance whose structure is known or a substance whose structure is unknown. Furthermore, it may be a mixture or the like of various compounds, and may be a substance, a compound, or a composition belonging to any field such as the medical field, the industrial field, the food field, or the like. Examples of the target substance belonging to the medical field include proteins, antibodies, beads with antibodies, tumor markers, and the like. On the other hand, examples of the target substance belonging to the industrial field include metal nanoparticles, carbon nanotubes, magnetic fluid, nanosilica, crystalline zirconia and the like. Examples of the target substance belonging to the food field include agricultural products and processed products thereof.

Hereinafter, the analysis method of the present embodiment will be described in detail. In the following description, a case where luminescent dye molecules are disposed in the first component disposing step and a target substance is further disposed in the reaction field in the second component disposing step will be described as an example. However, the analysis method of the present embodiment is not limited to the above-described method.

### (First Component Disposing Step)

In the first component disposing step, the luminescent dye molecules are disposed in a reaction field of a plate including the reaction field for allowing the target substance and the luminescent dye molecules to interact with each other.

The plate used in this step may have the reaction field, and the number of reaction fields may be one, or two or more. From the viewpoint of analyzing a plurality of target substances or analyzing a target substance using a plurality of luminescent dye molecules, it is preferable that one plate has a plurality of reaction fields. When the plate has a plurality of reaction fields, these are preferably disposed at intervals. The plate may have a flat plate shape or may have recesses and projections in accordance with the shape of the reaction field. The material, size, shape, and the like of the plate are appropriately selected according to the use of analysis, the types of the luminescent dye molecules and the target substance, and the like.

When the plate has a plurality of reaction fields, the positions of the reaction fields are preferably set at intervals so that adjacent reaction fields do not contact with each other. The interval is appropriately selected according to the size of the reaction field, the types of the luminescent dye molecule and the target substance, and the like. In a case where the first component disposing step and the second component disposing step are performed by a machine (for example, an ink jet apparatus or the like), a mark (formation of an uneven structure or marking) or the like indicating the position of each reaction field may not be formed on the plate. On the other hand, when a mark (formation of an uneven structure or marking) indicating the position of each reaction field is formed on the plate, it is easy to accurately dispose the target substance or the luminescent dye molecule at a desired position (reaction field) when the first component disposing step or the second component disposing step is performed.

In addition, when each reaction field is formed in a concave shape, or a partition wall part is disposed around each reaction field, the target substances and the luminescent dye molecules disposed in adjacent reaction fields are hardly mixed, and more accurate analysis is easily performed. In addition, for example, in a case where the water repellent treatment portion is disposed around the reaction fields, the target substance and the luminescent dye molecule in the adjacent reaction fields are less likely to be mixed, and thus it is easy to perform more accurate analysis. In the present embodiment, a plate in which a plurality of wells are regularly disposed is used. In the plate including such wells, since the wells (reaction fields) are physically separated from each other by the partition walls, the target substances and the luminescent dye molecules in the adjacent reaction fields are less likely to be mixed, and accurate analysis can be easily performed.

Here, the number of reaction fields included in one plate is appropriately selected according to the type of the target substance to be analyzed, the type of the luminescent dye molecule, and the like. The number of reaction fields is not particularly limited, but the greater the number, the more numerous and multidimensional data can be acquired, and more precise analysis can be performed.

The method for disposing the first component (luminescent dye molecules in the present embodiment) in each reaction field is not particularly limited, and is appropriately selected according to the type, physical properties, and the like of the first component. Examples of the method for disposing the first component include application by an ink jet apparatus, application by a dispenser, disposing of a carrier for carrying the first component, direct fixation of the first component to the reaction field, and the like. Among these, the inkjet method is particularly preferable. According to the ink jet method, it is possible to efficiently dispose respective liquid first components (luminescent dye molecules) in a large number of regions (reaction fields) to form the reaction fields. This makes it possible to acquire a large amount of data.

When the plate has a plurality of reaction fields, the same first component (luminescent dye molecule) may be disposed in all of the plurality of reaction fields, or a plurality of types of first components (luminescent dye molecules) may be disposed in the same reaction field. Further, first components (luminescent dye molecules) including different compositions may be disposed in two or more reaction fields. When different types of first components (luminescent dye molecules) are disposed in different reaction fields, respectively, a plurality of types of interactions between the luminescent dye molecules and the target substance occur, and the target substance can be analyzed in more detail.

### (First Signal Information Acquiring Step)

In the first signal information acquiring step, first signal information is acquired from the plate in which the first component is disposed in the reaction field. The first signal information obtained in this step is not particularly limited as long as it is information useful for the analysis described below. As described above, the luminescent dye molecules emit a plurality of types of light in response to a single excitation light. Therefore, specific excitation light (excitation light having a single wavelength) may be irradiated, and the intensity and wavelength (light emission information) of light emitted by the luminescent dye molecule may be acquired as the first signal information. Furthermore, for example, a change over time in the spectral distribution of light emitted by the luminescent dye molecules when irradiated with specific excitation light, or a change in chromaticity over time may be acquired as the first signal. The information acquired in the first signal information acquiring step may be of one type or two or more types.

When the intensity or wavelength of light emitted by the luminescent dye molecule is acquired, excitation light having a single wavelength may be applied, and the emission intensity or emission wavelength of the luminescent dye molecule may be acquired using a general spectrophotometer or the like. Furthermore, when the spectral distribution change of the luminescent dye molecule is acquired, excitation light having a single wavelength may be applied only for a short time, and light emitted by the luminescent dye molecule in response thereto may be acquired continuously or intermittently with a spectrophotometer or the like.

Furthermore, in the case of acquiring a chromaticity change of light emitted by the luminescent dye molecules, excitation light having a single wavelength may be applied only for a short time, and an image of the light emitted by the luminescent dye molecules upon receipt of the excitation light may be acquired with a CCD camera, a CMOS camera, or the like. The chromaticity is specified from the obtained image, so that data on the change in chromaticity over time can be acquired.

### (Second Component Disposing Step)

In the second component disposing step, the other one (which is the target substance in the present embodiment) of the luminescent dye molecule and the target substance is disposed in the reaction field from which the first signal information has been acquired. When the above-described plate has a plurality of reaction fields, the second components (the target substances in the present embodiment) including different compositions may be disposed in some or all of the reaction fields. On the other hand, the second components (target substances) including the same composition may be disposed in all the reaction fields.

Note that the method of disposing the second component (target substance) is not particularly limited, and is appropriately selected depending on the type and properties of the second component. The method may be the same as the method for disposing the first component described above. Furthermore, in the second component disposing step, the second component may also be disposed in a region where the above-described first component is not disposed.

### (Second Signal Information Acquiring Step)

In the second signal information acquiring step, second signal information is acquired from the plate in which the first component is disposed. The second signal information acquired in this step is not particularly limited as long as it is information useful for analysis in the analyzing step described later. In general, it is preferable to acquire the second signal information in the same manner as the information acquired in the first signal information acquiring step.

### (Analyzing Step)

In the analyzing step, the first signal information acquired in the first signal information acquiring step and the second signal information acquired in the second signal information acquiring step are compared to analyze the target substance. Specifically, data (hereinafter, also referred to as “analysis data”) is obtained by subtracting the first signal information from the second signal information. Then, the state or the like of the target substance is analyzed on the basis of the size, value, or the like of the analysis data. Note that the method of analyzing the analysis data in this step is appropriately selected according to the purpose, the type of the analysis data, and the like.

For example, for an ideal target substance, standard data may be prepared in advance by performing steps similar to the first component disposing step, the first signal information acquiring step, the second component disposing step, the second signal information acquiring step, and the like, and the state, structure, and the like of the target substance may be specified by comparing the standard data with the analysis data. In addition, when the target substance is composed of a plurality of components or when a plurality of parameters are involved (for example, quality or deliciousness of food), standard data in a case where the target substance is in a good state and standard data in a case where the target substance is in a bad state may be created and compared.

Note that in the case of performing the analysis, the standard data and the analysis data may be simply compared with each other, but for example, the result of the comparison between the standard data and the analysis data may be converted into a distance matrix and analyzed with a heat map (without weighting), the distance matrix may be subjected to principal component analysis (weighting emphasizing anisotropy, also referred to as PCA), analysis by DL (weighting emphasizing isotropy), or the like.

On the other hand, the standard data may be a learned model or the like generated in advance by machine learning. The learned model can be created by, for example, a machine learning step to be described later, but the learned model to be used is not limited to a model created in the machine learning step to be described later. Using the learned model, more appropriate analysis can be performed on the target substance.

In a case where the learned model is referred to, by applying the above-described analysis data to the learned model, it is possible to determine (predict) whether the target substance has a desired structure, how much the target substance includes a predetermined structure, whether the target substance is in a good state, and the like from the accumulated data or the like. Note that the prediction result may be obtained as, for example, classification, regression, clustering, abnormality detection (outlier detection), or the like.

### (Machine Learning Step)

The analysis method of the present embodiment may further include a learning step of performing machine learning of the above-described first signal information and the second signal information to generate a learned model.

For example, in the machine learning step, a plurality of prediction models are constructed based on the above-described difference between the second signal information and the first signal information (analysis data). Then, the results of the plurality of prediction models are combined to create a learned model that can predict information on the target substance (e.g., structure and amount).

In a case where the structure or amount of a target substance is known in advance, for example, the prediction model can be constructed by performing machine learning in which the features of analysis data are explanatory variables and the structure, amount, or the like of the target substance is an objective variable. As the explanatory variables, numerical values representing the features of the above-described analysis data and numerical values calculated from the numerical values can be used. When the first signal information and the second signal information are spectral distributions, the intensity of light at each wavelength or the like can be adopted as an explanatory variable. On the other hand, the objective variable can be appropriately selected according to the purpose of analysis and is not limited to the structure or amount of the target substance, but any other variable related to the target substance may be used.

The machine learning performed in this step may be supervised learning or may be unsupervised learning. Note that supervised learning refers to a learning method of learning a “relationship between an input and an output” from learning data with a correct label. Unsupervised learning is a learning method of learning a “structure of a data group” from learning data without a correct label.

Alternatively, the machine learning may be reinforcement learning, deep learning, or deep layer reinforcement learning. Note that reinforcement learning refers to a learning method of learning an “optimal action sequence” by trial and error. Deep learning refers to a learning method of learning, from a large amount of data, features included in the data step by step more deeply (in deeper layers). The deep reinforcement learning refers to a learning method in which reinforcement learning and deep learning are combined.

A general analysis method (algorithm) can be applied to the machine learning. For the machine learning, for example, a prediction model constructed by an analysis method selected from linear regression (multiple regression analysis, partial least squares (PLS) regression, LASSO regression, Ridge regression, principal component regression (PCR), and the like), random forest, decision tree, support vector machine (SVM), support vector regression (SVR), neural network, discriminant analysis, and the like can be applied.

### (Modification Example)

It has been described above that the plate including the reaction field configured to cause the target substance and the luminescent dye molecules to interact with each other is used, and the target substance and the luminescent dye molecules are disposed in the reaction field to perform analysis. Provided that instead of the luminescent dye molecules, the luminescent substance carrier described above or the state sensing ink may be disposed. Furthermore, a fluorescence intensity measurement cell may be used in place of the plate. In this case, the first signal information acquiring step and the second signal information acquiring step may be performed by a known fluorescence measurement device. Further, in the case of using the state sensing ink or the like, a desired base material (for example, paper or the like) may be used instead of the above-described plate, and the state sensing ink or the target substance may be printed thereon in the first component disposing step or the second component disposing step.

### (Effect of Analysis Method)

In the analysis method using the luminescent dye molecule, the luminescent dye molecule and the target substance are caused to interact with each other to acquire the first signal information and the second signal information. Then, by analyzing the first signal information and the second signal information, various information on the target substance can be obtained.

In addition, since the above-described luminescent dye molecule is used, it is possible to acquire a large amount of data by allowing the target substance and the luminescent dye molecule to appropriately interact with each other. Therefore, the target substance can be analyzed in detail.

Furthermore, according to this analysis method, it is possible to construct a novel method for simply describing, that is, sensing a complicated state of a substance or gas serving as a specimen.

### EXAMPLES

### 1. Example 1

### 1-1. Synthesis of Luminescent Dye Molecules 1 to 16

All reactions were performed under a nitrogen atmosphere in oven-dried glassware unless otherwise noted. All chemical products were purchased from Aldrich or TCI or Kanto Chemical and used as is without further purification.

### (1) Synthesis of Monomer 1

Based on the following reaction formula, a monomer 1 having a main chain containing a phosphate ester and a luminophore bonded to the main chain was synthesized via intermediates 1 to 6.

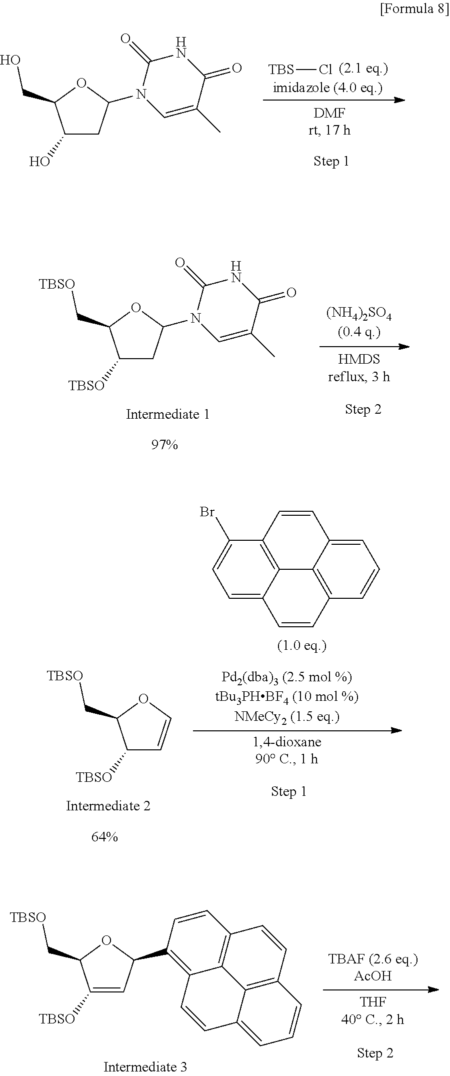

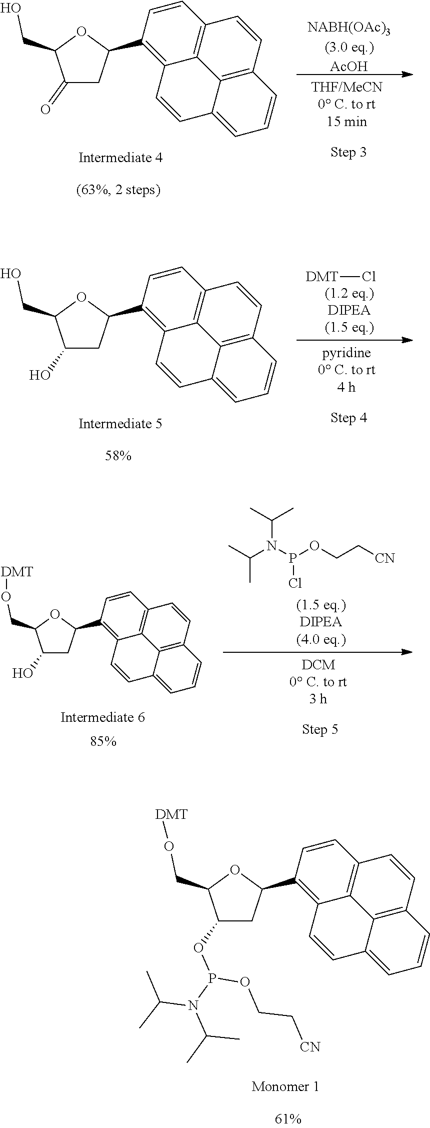

### Synthesis of Intermediate 1

Thymidine (15.0 g, 61.9 mmol) and imidazole (16.9 g, 248 mmol) were dissolved in DMF (124 mL), tert-butyldimethylsilylchloride (19.6 g, 130 mmol) was added, and the mixture was stirred at room temperature for 17 hr. To the reaction mixture was added water, and the mixture was partitioned and extracted with ethyl acetate. The obtained organic phase was dried with magnesium sulphate, and the solvent was distilled off to obtain the desired intermediate 1 as a colorless solid (28.3 g, 97%).

### Synthesis of Intermediate 2

The intermediate 1 (28.3 g, 60.1 mmol) and ammonium sulphate (12.7 g, 96.2 mmol) were dissolved in hexamethyldisilazane (314 mL, 1.50 mol), and the mixture was heated under reflux for 3 hours. The resulting crude product was purified by silica gel column chromatography to obtain the desired intermediate 2 as a brown liquid (13.2 g, 64%).

### Synthesis of Intermediate 4

A mixture of intermediate 2 (10.1 g, 29.3 mmol), 1-bromopyrene (8.24 g, 29.3 mmol), tris (dibenzylideneacetone) dipalladium (0) (671 mg, 733 μmol), tri-tertbutylphosphonium tetrafluoroborate (850 mg, 2.93 mmol), dicyclohexylmethylamine (9.35 mL, 44.0 mmol), 1,4-dioxane (100 mL) was heated at 90° C. for 1 hour. Water was added to stop the reaction, and liquid separation and extraction were performed with ethyl acetate. The obtained organic phase was dried over magnesium sulfate, and the solvent was distilled off to give a crude product containing intermediate 3, which was used as is in the next reaction.

To the crude product containing the intermediate 3 were added THF 100 mL, 1M tetrabutylammonium fluoride in THF (117 mL, 117 mmol), and ethyl acetate (6.74 mL, 117 mmol), and the mixture was stirred at 40° C. for 2 hr. Water was added to stop the reaction, and liquid separation and extraction were performed with ethyl acetate. The obtained crude product was purified by silica gel column chromatography to obtain the desired intermediate 4 as a light brown solid (5.87 g, 63%).

### Synthesis of Intermediate 5

A solution of sodium triacetylborate (11.8 g, 55.8 mmol) and acetic acids (7.87 mL, 138 mmol) in acetonitrile 93 mL was cooled to 0° C., and the solution of the intermediate 4 (5.87 g, 18.6 mmol) in THF (62 mL) was added dropwise. After completion of the dropwise addition, the mixture was warmed to room temperature and stirred for 15 min, and water was added to stop the reaction. Liquid separation and extraction were performed with ethyl acetate, the obtained organic phase was dried over magnesium sulfate, and the solvent was distilled off to obtain a crude product. Purification by silica gel column chromatography and reversed-phase HPLC afforded the desired intermediate 5 as a colorless solid (3.44 g, 58%).

### Synthesis of Intermediate 6

A mixture of the intermediate 5 (3.44 g, 10.8 mmol), 4,4′-dimethoxytritylchloride (4.40 g, 13.0 mmol), ethyldiisopropylamine (2.82 mL, 16.2 mmol) and dehydrated pyridine (54 mL) was stirred at room temperature for 4 hours, and then methanol was added to stop the reaction. The solvent was distilled off and the obtained crude product was purified by silica gel column chromatography to obtain the desired intermediate 6 as a colorless viscous solid (5.71 g, 85%).

### Synthesis of Monomer 1

To a mixture of intermediate 6 (5.71 g, 9.20 mmol), ethyldiisopropylamine (6.42 mL, 36.8 mmol), and dehydrated dichloromethane (92 mL) was added dropwise 2-cyanoethyldiisopropylchlorophosphoroamidite (3.08 mL, 13.8 mmol) at 0° C. After heating to room temperature and stirring for 3 hours, the solvent was distilled off to obtain a crude product. The target monomer 1 was obtained as a colorless solid by purification by silica gel column chromatography (4.64 g, 61%).

### (2) Preparation of Monomer 2

Monomer 2, a reagent of the structure shown below, was purchased from Glen Research (Sterling, Va).

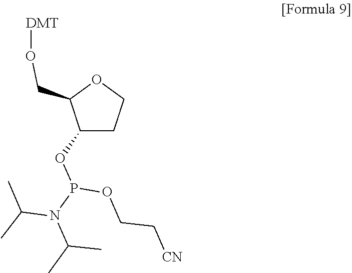

### (3) Synthesis of Luminescent Dye Molecules 1 to 16

According to a conventional method, as illustrated in Table 2 below, 16 types of oligonucleotides (Seq. 1 to 16) having a mixed sequence of the monomer 1 and the monomer 2 were synthesized. DNA synthesis reagents were purchased from Glen Research (Sterling, Va). In addition, all of the oligonucleotides were synthesized with a DNA/RNA synthesizer NTS Terminator manufactured by Nippon Technoservice Co., Ltd. using a standard protocol for a phosphoramidites based coupling procedure. Each of the luminescent dye molecule-carrying bodies obtained by the automatic synthesis was reacted with ammonium water at room temperature for 2 hours, cut out from the particulate carrier, the solvent was dried to dryness with a centrifugal drying apparatus, and then ultrapure water was added, thereby obtaining first components 1 to 16 containing luminescent dye molecules 1 to 16, respectively. It was confirmed that the luminescent dye molecules 1 to 15 emit fluorescence and excimer light emission in response to specific excitation light (light of wavelengths 350 nm). Note that the luminescent dye molecule 16 did not exhibit fluorescence or excimer light emission. The proportion of the β-form of deoxyribose in the table was obtained as the equation: {(the number of β-deoxyribose in the luminescent dye molecules)/(the number of deoxyribose in the luminescent dye molecules)}×100 [%].

TABLE 1

| | Luminescent dye molecule | | | | | | | | | | | | | | | | |
|---|---|---|---|---|---|---|---|---|---|---|---|---|---|---|---|---|---|
| | | 1 | 2 | 3 | 4 | 5 | 6 | 7 | 8 | 9 | 10 | 11 | 12 | 13 | 14 | 15 | 16 |
| Monomer | 5′ | 1 | 1 | 1 | 1 | 1 | 1 | 1 | 1 | 2 | 2 | 2 | 2 | 2 | 2 | 2 | 2 |
| sequence | 3′ | 1 | 1 | 1 | 1 | 2 | 2 | 2 | 2 | 1 | 1 | 1 | 1 | 2 | 2 | 2 | 2 |
| | | 1 | 1 | 2 | 2 | 1 | 1 | 2 | 2 | 1 | 1 | 2 | 2 | 1 | 1 | 2 | 2 |
| | | 1 | 2 | 1 | 2 | 1 | 2 | 1 | 2 | 1 | 2 | 1 | 2 | 1 | 2 | 1 | 2 |

| | | | | | | | | | | | | | | | | |
|---|---|---|---|---|---|---|---|---|---|---|---|---|---|---|---|---|
| Deoxyribose | 100 | 100 | 100 | 100 | 100 | 100 | 100 | 100 | 100 | 100 | 100 | 100 | 100 | 100 | 100 | — |
| β-form | | | | | | | | | | | | | | | | |
| proportion (%) | | | | | | | | | | | | | | | | |
| Number of | 4 | 4 | 4 | 2 | 3 | 2 | 2 | 1 | 3 | 2 | 2 | 1 | 1 | 1 | 1 | 0 |
| chromophores or | | | | | | | | | | | | | | | | |
| luminophores | | | | | | | | | | | | | | | | |
| Natural | 0 | 0 | 0 | 0 | 0 | 0 | 0 | 0 | 0 | 0 | 0 | 0 | 0 | 0 | 0 | 0 |
| nucleic acid base | | | | | | | | | | | | | | | | |
| proportion (%) | | | | | | | | | | | | | | | | |

### 1-2. Luminescent Dye Molecule Disposing Step

A 96-well microplate in which wells with opening diameters of 7 mm were disposed at intervals of 9 mm in 12 columns and × 8 rows was prepared. The luminescent dye molecules 1 to 16 were placed in the 96-well microplate in an amount of 100u 1 each with an automatic dispenser (NichiMart CUBE, manufactured by NICHIRYO Co., Ltd) to form a plurality of reaction fields.

### 1-3. First Signal Information Acquiring Step

A 96-well microplate on which luminescent dye molecules were placed was irradiated with excitation light (wavelength: 350 nm), and the fluorescence spectrum was obtained as first signal information.

### 1-4. Target Substance Disposing Step

In the 96-well microplate after the first signal information acquiring step, 20 μl of each of the three types of soft drinks (target substances 1 to 3) was placed by a method similar to the above.

### 1-5. Second Signal Information Acquiring Step

The fluorescence spectra obtained when the 96-well microplate in which the first components and the second components were disposed was irradiated with the excitation light (wave length 350 nm) were acquired as the second signal information.

### 1-6. Analyzing Step

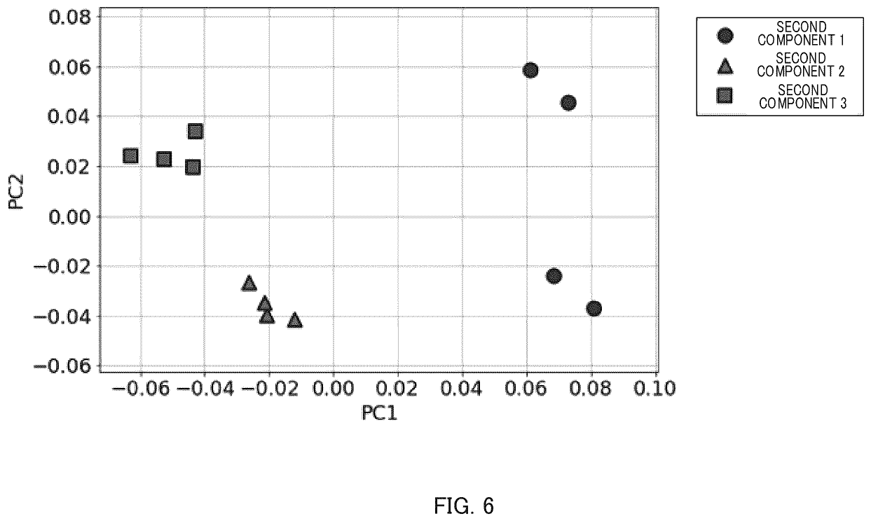

Analysis data was calculated by subtracting the first signal information acquired in the first signal information acquiring step from the second signal information acquired in the second signal information acquiring step. Then, when a principal component analysis was performed with the analysis data as an explanatory variable, as illustrated in FIG. 6, the second component (target substance) could be separated by type in the two-dimensional space in which principal components 1 and 2 were plotted.

### 1-7. Others

A pigment ink using, as a pigment dispersion liquid, the aqueous dispersion liquid of each luminescent dye molecule carrier obtained by the above-described automatic synthesis was prepared as follows, and it was confirmed that the same analysis method as described above could be performed thereon.

### [Production of Ink]

### Ink Raw Material

Triethylene glycol monobutyl ether (TEGmBE, manufactured by Tokyo Chemical Industry Co., Ltd) 5 parts by mass

2-Pyrrolidinone (manufactured by BASF) 8 parts by mass

Purified glycerin (manufactured by Kao Corporation) 2 parts by mass

Surfynol 440 (nonionic surfactant, manufactured by AIRPRODUCTS) 1,4,7,9tetramethyl-5decyne-4,7diol) 0.5 parts by mass

1,2-Hexanediol (manufactured by DEGUSSA AG) 1 parts by mass

Triethanolamine (manufactured by Konishi Co., Ltd) 0.8 parts by mass

Megaface 444 (nonionic surfactant, manufactured by DIC Corporation) (oxide adduct of perfluoroalkylethylene) 0.2 parts by mass

Polyurethane A (solid content) 0.5 parts by mass

AQUACER552 1 part by mass (solid content)

Pure water remaining amount (here, the “remaining amount” in the amount of pure water added means that the amount of pure water is adjusted so that the total of the parts by mass of the blended raw materials becomes 100 parts by mass)

The above components in the above proportions were placed in a 100 ml plastic container and stirred for 1 hour. Then, 20 parts of luminescent dye molecule 1 and carrier aqueous dispersion were added, and the mixture was further stirred for 1 hour to prepare a pigment ink composition.

### [Inkjet Ejection Evaluation Test]

The ejection evaluation was performed using the ink composition prepared by the above-described method. For ejection evaluation, printing was performed on photo paper (glossy) (HP Advance Photo Paper, manufactured by Hewlett-Packard Company), which is paper exclusively for inkjet printing, using a commercially available thermal-jet-type inkjet printer (Photosmart D5360, manufactured by Hewlett-Packard Company). As a result, it was confirmed that the ink composition prepared by the above-described method could be ejected by inkjet.

### [Preservability]

It was also confirmed that ink jet discharge was possible as described above even after storage at room temperature for one month.

### 2. Example 2

### 2-1. Synthesis of Luminescent Dye Molecules 1 to 75

### (1) Synthesis of Monomer 3

Based on the following reaction formula, a monomer 3 including a main chain containing a phosphate ester and a luminophore bonded to the main chain was synthesized via intermediates 7 to 10.

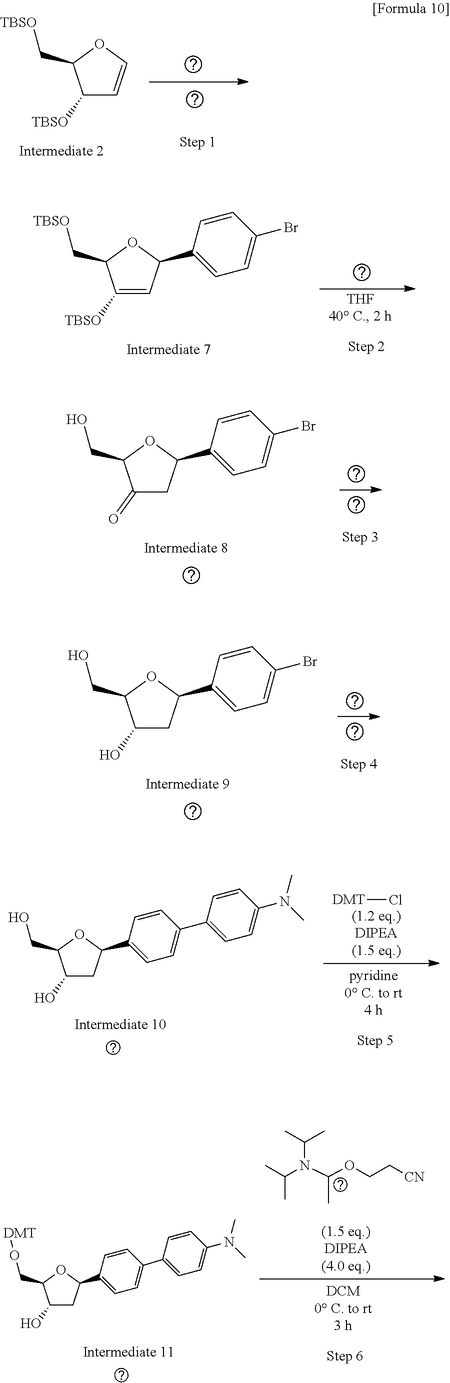

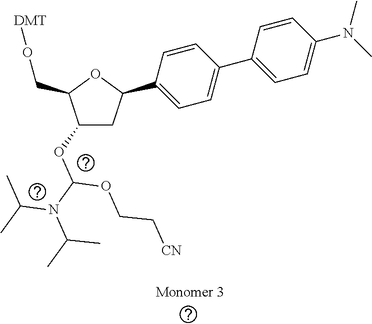

### Synthesis of Intermediate 8

A mixture of the intermediate 2 (2.00 g, 5.80 mmol), 1,4-dibromobenzene (8.24 g, 29.0 mmol), tris (dibenzylideneacetone) dipalladium (0) (133 mg, 145 μmol), tri-tert-butylphosphonium tetrafluoroborate (168 mg, 580 μmol), dicyclohexylmethylamine (1.85 mL, 8.70 mmol), 1,4-dioxane (29 mL) was heated at 90° C. for 3 hours. Water was added to stop the reaction, and liquid separation and extraction were performed with ethyl acetate. The obtained organic phase was dried over magnesium sulfate, and the solvent was distilled off to obtain a crude product containing the intermediate 7, which was used as it was for the next reaction.

To the crude product containing the intermediate 7 were added THF 29 mL, 1M tetrabutylammonium fluoride THF solution (23.2 mL, 23.2 mmol), and acetic acid (1.32 mL, 23.2 mmol), and the mixture was stirred at 40° C. for 2 hr. Water was added to stop the reaction, and liquid separation and extraction were performed with ethyl acetate. The crude product obtained by distilling off the solvents was purified by silica gel column chromatography to obtain the target intermediate 8 as a yellowish brown oil (1.08 g, 69%).

### Synthesis of Intermediate 9

A solution of sodium triacetylborate (2 52 g, 11.9 mmol) and acetic acid (1.82 mL, 31.8 mmol) in acetonitrile 20 mL was cooled to 0° C., and a solution of the intermediate 8 (1.08 g, 3.98 mmol) in THF (13 mL) was added dropwise. After completion of the dropwise addition, the mixture was warmed to room temperature and stirred for 2 hours and 30 minutes, and then water was added to stop the reaction. Liquid separation and extraction were performed with ethyl acetate, the obtained organic phase was dried over magnesium sulfate, and the solvent was distilled off to obtain a crude product. After washing with heptane, purification by recrystallization from ethyl acetate gave the desired intermediate 9 as a colorless solid (481 mg, 44%).

### Synthesis of Intermediate 10

A mixture of intermediate 9 (1.00 g, 3.66 mmol), N,N-dimethyl-4v (4,4,5,5-tetramethyl-1,3,2-dioxaborolan-2-yl) aniline (905 mg, 3.66 mmol), bis(dibenzylideneacetone) palladium (0) (106 mg, 184 μmol), ethyldiisopropylamine 2-dicyclohexylphosphino-2′,4′,6′-triisopropylbiphenyl (175 mg, 367 μmol), potassium phosphate (2.33 g, 367 μmol), N,N-dimethylformamide (33 mL), and water (4 mL) was stirred at 90° C. for 1 hour, then water was added to stop the reaction. The resultant was subjected to separation and extraction with dichloromethane. The crude product obtained by distilling off the solvents was purified by silica gel column chromatography to obtain the desired intermediate 10 as a colorless solid (1.10 g, 96%).

### Synthesis of Intermediate 11

A mixture of the intermediate 10 (1.10 g, 3.52 mmol), 4,4′-dimethoxytritylchloride (1.32 g, 3.90 mmol), ethyldiisopropylamine (0.92 mL, 5.29 mmol), and dehydrated pyridine (17.5 mL) was stirred at room temperature for 4 hours, and then methanol was added thereto to stop the reaction. The solvents were distilled off and the obtained crude product was purified by silica gel column chromatography to obtain the desired intermediate 11 as a yellow viscous solid (2.89 g, 82%).

### Synthesis of Monomer 3

To a mixture of intermediate 11 (1.23 g, 2.00 mmol), ethyldiisopropylamine (1.39 mL, 8.00 mmol) and dehydrated dichloromethane (80 mL) was added dropwise 2-cyanoethyldiisopropylchlorophosphoroamidite (468 μL, 2.10 mmol) at room temperature. After stirring for 2 hours, the solvent was distilled off to obtain a crude product. Purification by silica gel column chromatography gave the target monomer 3 as a yellow viscous solid (1.53 g, 94%).

### (2) Preparation of Monomer 4

Monomer 4, a reagent of the structure shown below, was purchased from Glen Research (Sterling, Va). The thymidine contained in the monomer 4 is a natural nucleic acid base.

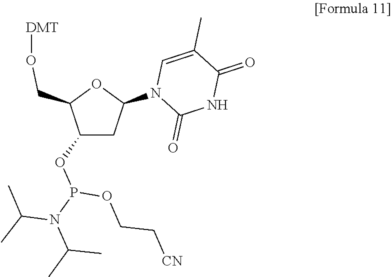

### (3) Synthesis of Monomer 5

The monomer 5 was synthesized according to a non-patent literature (J. Am. Chem. Soc. 1996, 118,7671-7678). Note that monomer 5 is a structural isomer of the above-described monomer 1 and is a monomer in which the sugar structure is an α-form.

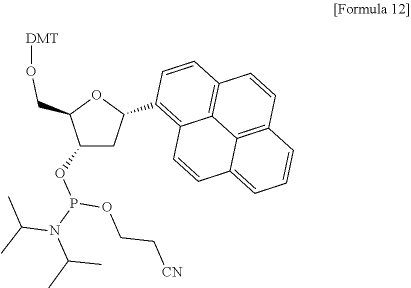

### (4) Synthesis of Monomer 6

The monomer 6 was synthesized according to a non-patent literature (Tetrahedron Lett. 1999, 40, 419-422). Note that the monomer 5 is a monomer of peptide nucleic acid (PNA).

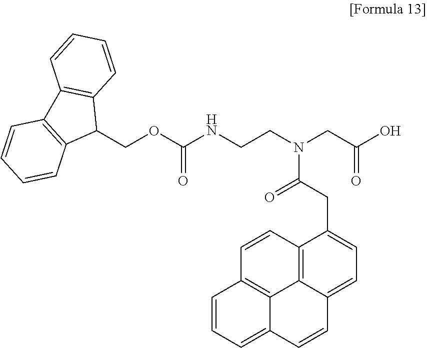 [SMILES: O=C(O)CN(CCNC(=O)OCC1c2ccccc2-c2ccccc21)C(=O)Cc1ccc2ccc3cccc4ccc1c2c34]

### (5) Synthesis of Monomer 7

The monomer 6 was synthesized according to the non-patent literature (Tetrahedron Lett. 1999, 40, 419-422). Note that the monomer 5 is a monomer of peptide nucleic acid (PNA).

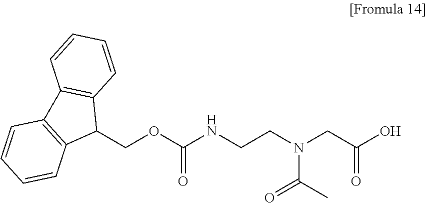 [SMILES: CC(=O)N(CCNC(=O)OCC1c2ccccc2-c2ccccc21)CC(=O)O]

### (7) Synthesis of Luminescent Dye Molecules 1 to 75

### (7-1) Preparation of Luminescent Dye Molecules 1 to 16

Luminescent dye molecules 1 to 16 were prepared in the same manner as in Example 1 described above.

### (7-2) Synthesis of Luminescent Dye Molecules 1 and 17 to 31

As illustrated in the following Table 2, 16 types (Seq1 and 17 to 31) of mixed sequence oligonucleotides with the above-described Monomer 1 and aniline-containing Monomer 3 were synthesized by a method similar to the above-described method of synthesizing the luminescent dye molecules 1 to 16. It was confirmed that the luminescent dye molecules 1 and 17 to 31 emit fluorescence and exciplex light emission in response to specific excitation light (light having wavelengths 350 nm).

TABLE 2

| | Luminescent dye molecule | | | | | | | | | | | | | | | | |
|---|---|---|---|---|---|---|---|---|---|---|---|---|---|---|---|---|---|
| | | 1 | 17 | 18 | 19 | 20 | 21 | 22 | 23 | 24 | 25 | 26 | 27 | 28 | 29 | 30 | 31 |
| Monomer | 5′ | 1 | 1 | 1 | 1 | 1 | 1 | 1 | 1 | 3 | 3 | 3 | 3 | 3 | 3 | 3 | 3 |
| sequence | 3′ | 1 | 1 | 1 | 1 | 3 | 3 | 3 | 3 | 1 | 1 | 1 | 1 | 3 | 3 | 3 | 3 |
| | | 1 | 1 | 3 | 3 | 1 | 1 | 3 | 3 | 1 | 1 | 3 | 3 | 1 | 1 | 3 | 3 |
| | | 1 | 3 | 1 | 3 | 1 | 3 | 1 | 3 | 1 | 3 | 1 | 3 | 1 | 3 | 1 | 3 |

| | | | | | | | | | | | | | | | | |
|---|---|---|---|---|---|---|---|---|---|---|---|---|---|---|---|---|
| Deoxyribose | 100 | 100 | 100 | 100 | 100 | 100 | 100 | 100 | 100 | 100 | 100 | 100 | 100 | 100 | 100 | 100 |
| β-form | | | | | | | | | | | | | | | | |
| proportion (%) | | | | | | | | | | | | | | | | |
| Number of | 4 | 4 | 4 | 4 | 4 | 4 | 4 | 4 | 4 | 4 | 4 | 4 | 4 | 4 | 4 | 4 |
| chromophores or | | | | | | | | | | | | | | | | |
| luminophores | | | | | | | | | | | | | | | | |
| Natural | 0 | 0 | 0 | 0 | 0 | 0 | 0 | 0 | 0 | 0 | 0 | 0 | 0 | 0 | 0 | 0 |
| nucleic acid base | | | | | | | | | | | | | | | | |
| proportion (%) | | | | | | | | | | | | | | | | |

### (7-3) Synthesis of Luminescent Dye Molecules 1 and 32 to 45

As illustrated in Table 3 below, 15 types of oligonucleotides (Seq. 1 and Seq. 32 to 45) including a mixed sequence of the monomer 1 and the thymidine-containing monomer 4 described above were synthesized by a method similar to the method for synthesizing the luminescent dye molecules 1 to 16 described above. In addition, it was confirmed that the luminescent dye molecules 1 and 32 to 45 emit fluorescence and excimer light emission in response to specific excitation light (light of wavelengths 350 nm).

TABLE 3

| | Luminescent dye molecule | | | | | | | | | | | | | | | |
|---|---|---|---|---|---|---|---|---|---|---|---|---|---|---|---|---|
| | | 1 | 32 | 33 | 34 | 35 | 36 | 37 | 38 | 39 | 40 | 41 | 42 | 43 | 44 | 45 |
| Monomer | 5′ | 1 | 1 | 1 | 1 | 1 | 1 | 1 | 1 | 4 | 4 | 4 | 4 | 4 | 4 | 4 |
| sequence | 3′ | 1 | 1 | 1 | 1 | 4 | 4 | 4 | 4 | 1 | 1 | 1 | 1 | 4 | 4 | 4 |
| | | 1 | 1 | 4 | 4 | 1 | 1 | 4 | 4 | 1 | 1 | 4 | 4 | 1 | 1 | 4 |
| | | 1 | 4 | 1 | 4 | 1 | 4 | 1 | 4 | 1 | 4 | 1 | 4 | 1 | 4 | 1 |

| | | | | | | | | | | | | | | | |
|---|---|---|---|---|---|---|---|---|---|---|---|---|---|---|---|
| Deoxyribose | 100 | 100 | 100 | 100 | 100 | 100 | 100 | 100 | 100 | 100 | 100 | 100 | 100 | 100 | 100 |
| β-form | | | | | | | | | | | | | | | |
| proportion (%) | | | | | | | | | | | | | | | |
| Number of | 4 | 3 | 3 | 2 | 3 | 2 | 2 | 1 | 3 | 2 | 2 | 1 | 2 | 1 | 1 |
| chromophores or | | | | | | | | | | | | | | | |
| luminophores | | | | | | | | | | | | | | | |
| Natural | 0 | 25 | 25 | 50 | 25 | 50 | 50 | 75 | 25 | 50 | 50 | 75 | 50 | 75 | 75 |
| nucleic acid base | | | | | | | | | | | | | | | |
| proportion (%) | | | | | | | | | | | | | | | |

### (7-4) Synthesis of Luminescent Dye Molecules 46 to 60

As illustrated in the following Table 4, 15 types of mixed sequence oligonucleotides (Seq. 46 to 60) of the above-described Monomer 5 containing an α-form sugar structure and the Monomer 2 were synthesized by a method similar to the method of synthesizing the above-described luminescent dye molecules 1 to 16. It was confirmed that the luminescent dye molecules 46 to 60 emit fluorescence and excimer light emission in response to specific excitation light (light of wavelength 350 nm).

TABLE 4

| | Luminescent dye molecule | | | | | | | | | | | | | | | |
|---|---|---|---|---|---|---|---|---|---|---|---|---|---|---|---|---|
| | | 46 | 47 | 48 | 49 | 50 | 51 | 52 | 53 | 54 | 55 | 56 | 57 | 58 | 59 | 60 |
| Monomer | 5′ | 5 | 5 | 5 | 5 | 5 | 5 | 5 | 5 | 2 | 2 | 2 | 2 | 2 | 2 | 2 |
| sequence | 3′ | 5 | 5 | 5 | 5 | 2 | 2 | 2 | 2 | 5 | 5 | 5 | 5 | 2 | 2 | 2 |
| | | 5 | 5 | 2 | 2 | 5 | 5 | 2 | 2 | 5 | 5 | 2 | 2 | 5 | 5 | 2 |
| | | 5 | 2 | 5 | 2 | 5 | 2 | 5 | 2 | 5 | 2 | 5 | 2 | 5 | 2 | 5 |

| | | | | | | | | | | | | | | | |
|---|---|---|---|---|---|---|---|---|---|---|---|---|---|---|---|
| Deoxyribose | 0 | 0 | 0 | 0 | 0 | 0 | 0 | 0 | 0 | 0 | 0 | 0 | 0 | 0 | 0 |
| β-form | | | | | | | | | | | | | | | |
| proportion (%) | | | | | | | | | | | | | | | |
| Number of | 4 | 3 | 3 | 2 | 3 | 2 | 2 | 1 | 3 | 2 | 2 | 1 | 2 | 1 | 1 |
| chromophores or | | | | | | | | | | | | | | | |
| luminophores | | | | | | | | | | | | | | | |
| Natural | 0 | 0 | 0 | 0 | 0 | 0 | 0 | 0 | 0 | 0 | 0 | 0 | 0 | 0 | 0 |
| nucleic acid base | | | | | | | | | | | | | | | |
| proportion (%) | | | | | | | | | | | | | | | |

### (7-5) Synthesis of Luminescent Dye Molecules 61 to 75

As shown in Table 5 below, the synthesis of mixed sequence PNA16 species (Seq. 61 to 75) with monomers 6 and 7 described above was performed. The PNA synthesis reagents were purchased from Sigma-Aldrich. In addition, all of the peptides were synthesized with an automatic peptide synthesizer SyroII (manufactured by Biotage) under atmosphere using the Fmoc method. Each of the luminescent dye molecule carriers obtained by the automatic synthesis was reacted with a mixed solution of TFA, triethylsilane, and water (90:2.5:2.5) for 2.5 hours at room temperature, then cut out from the carrier, and the solvent was dried by a centrifugal drying device, and then ultrapure water was added to obtain first components 61 to 75 containing respective luminescent dye molecules 61 to 75. It was confirmed that the luminescent dye molecules 1 and 61 to 75 emit fluorescence and excimer light emission in response to specific excitation light (light of wavelengths 350 nm).

TABLE 5

| | Luminescent dye molecule | | | | | | | | | | | | | | | |
|---|---|---|---|---|---|---|---|---|---|---|---|---|---|---|---|---|
| | | 61 | 62 | 63 | 64 | 65 | 66 | 67 | 68 | 69 | 70 | 71 | 72 | 73 | 74 | 75 |
| Monomer | 5′ | 6 | 6 | 6 | 6 | 6 | 6 | 6 | 6 | 7 | 7 | 7 | 7 | 7 | 7 | 7 |
| sequence | 3′ | 6 | 6 | 6 | 6 | 7 | 7 | 7 | 7 | 6 | 6 | 6 | 6 | 7 | 7 | 7 |
| | | 6 | 6 | 7 | 7 | 6 | 6 | 7 | 7 | 6 | 6 | 7 | 7 | 6 | 6 | 7 |
| | | 6 | 7 | 6 | 7 | 6 | 7 | 6 | 7 | 6 | 7 | 6 | 7 | 6 | 7 | 6 |

| | | | | | | | | | | | | | | | |
|---|---|---|---|---|---|---|---|---|---|---|---|---|---|---|---|
| Deoxyribose | — | — | — | — | — | — | — | — | — | — | — | — | — | — | — |
| β-form | | | | | | | | | | | | | | | |
| proportion (%) | | | | | | | | | | | | | | | |
| Number of | 4 | 3 | 3 | 2 | 3 | 2 | 2 | 1 | 3 | 2 | 2 | 1 | 2 | 1 | 1 |
| chromophores or | | | | | | | | | | | | | | | |
| luminophores | | | | | | | | | | | | | | | |
| Natural | 0 | 0 | 0 | 0 | 0 | 0 | 0 | 0 | 0 | 0 | 0 | 0 | 0 | 0 | 0 |
| nucleic acid base | | | | | | | | | | | | | | | |
| proportion (%) | | | | | | | | | | | | | | | |

2-2. Analysis with Light Emitting Element

### (1) Analysis Using Luminescent Dye Molecules 1 to 15

### Luminescent Dye Molecule Disposing Step

A plurality of 96-well microplates in which wells having 7 mm opening diameters were disposed at 9 mm intervals in 12 columns and×8 rows were prepared. The luminescent dye molecules 1 to 15 were placed in the 96-well microplate by an automatic dispensing device (NichiMart CUBE, manufactured by NICHIRYO Co., Ltd) in an amount of 100 μl for each of the target substances, to form a plurality of reaction fields.

### First Signal Information Acquiring Step

A fluorescence spectrum when a 96-well microplate in which the above-described luminescent dye molecules were disposed was irradiated with excitation light (wavelengths 350 nm) was acquired as first signal information.

### Target Substance Disposing Step

To the 96-well microplate after the first signal information acquiring step, 20 μl of 7 types and 95 brands of beverages (type I: 12 brands, type II: 23 brands, type III: 6 brands, type IV: 10 brands, type V: 20 brands, type VI: 13 brands, type VII: 11 brands) were disposed by the same method as described above.

### Second Signal Information Acquiring Step

The fluorescence spectra when the 96-well microplate in which the first components and the second components were disposed was irradiated with the excitation light (wave length 350 nm) were acquired as the second signal information.

### Analyzing Step

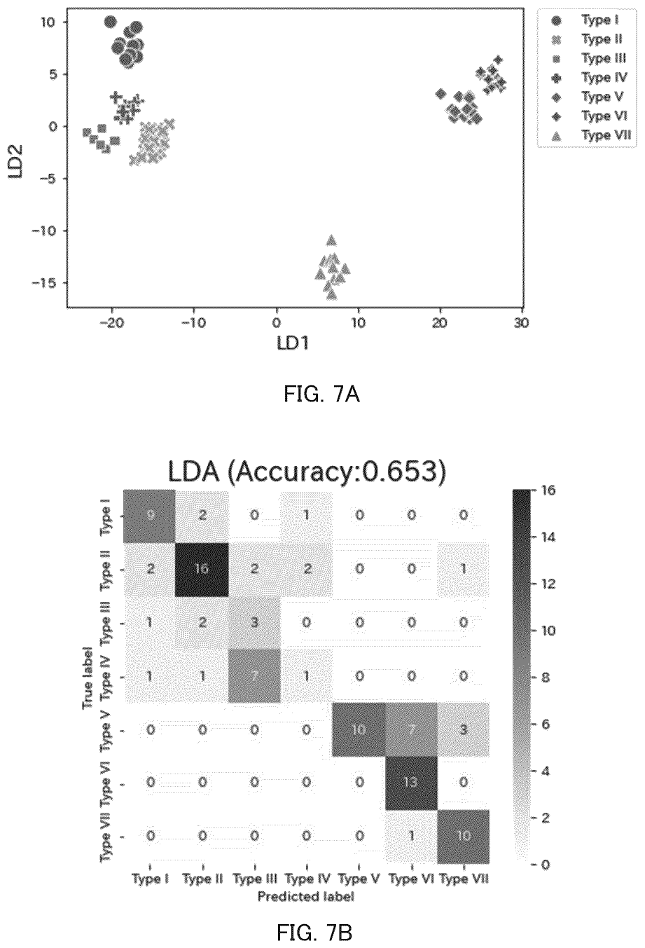

The analysis data was calculated by subtracting the first signal information acquired in the first signal information acquiring step from the second signal information acquired in the second signal information acquiring step. Then, learning was performed using the analysis data as explanatory variables and the classification data of each beverage as objective variables, and a discriminant model was created by linear discriminant analysis (LDA). The obtained linear discriminant analysis model plot is illustrated in FIG. 7A. Thereafter, calculation of a correct answer rate and creation of a confusion matrix were performed by cross-validation of 6 divisions, and generalization performance of the discriminant model was quantified. The confusion matrix is illustrated in FIG. 7B.

### Results

As illustrated in FIG. 7B, the discriminant model was able to classify the types of two brands of beverages with an accuracy of about 65%.

### (2) Analysis Using Luminescent Dye Molecules 1 and 17 to 31

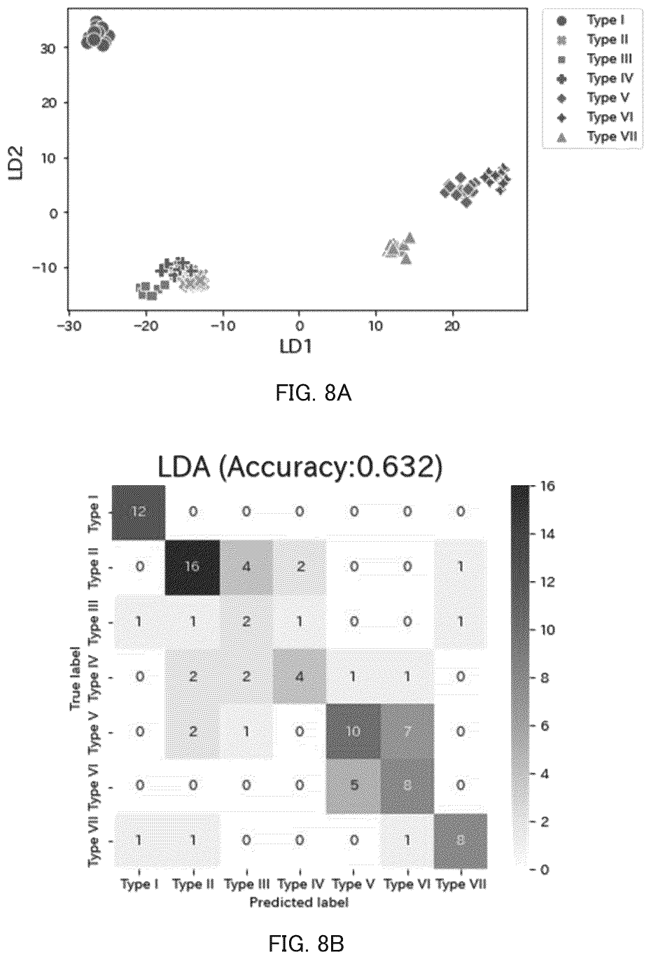

The luminescent dye molecules 1 and 17 to 31 (16 types) were used to perform the steps from the luminescent dye molecule disposing step to the analyzing step described above in the same manner. The obtained linear discriminant analysis model plot is illustrated in FIG. 8A. Then, calculation of a correct answer rate and creation of a confusion matrix were performed by cross-validation of six divisions, and generalization performance of the discriminant model was quantified. The confusion matrix is illustrated in FIG. 8B.

### Results

As illustrated in FIG. 8B, the discriminant model was able to classify the types of two brands of beverages with an accuracy of about 63%.

(3) Analysis Using luminescent dye molecules 1 to 15 and 17 to 31

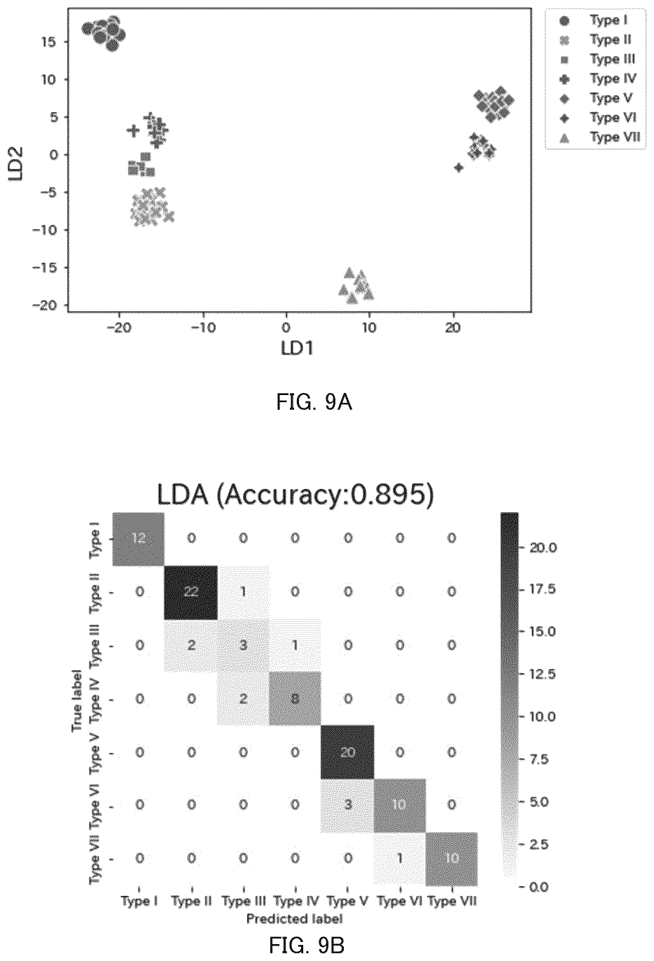

The luminescent dye molecules 1 to 15 and 17 to 31 (30 types) were used, and the steps from the luminescent dye molecule disposing step to the analyzing step were performed in the same manner. The linear discriminant analysis model plot thus obtained is illustrated in FIG. 9A. Then, calculation of a correct answer rate and creation of a confusion matrix were performed by cross-validation of six divisions, and generalization performance of the discriminant model was quantified. The confusion matrix is illustrated in FIG. 9B.

### Results

As illustrated in FIG. 9B, the discriminant model was able to classify the types of two brands of beverages with an accuracy of about 90%.

### (4) Analysis Using Luminescent Dye Molecules 1 and 32 to 45

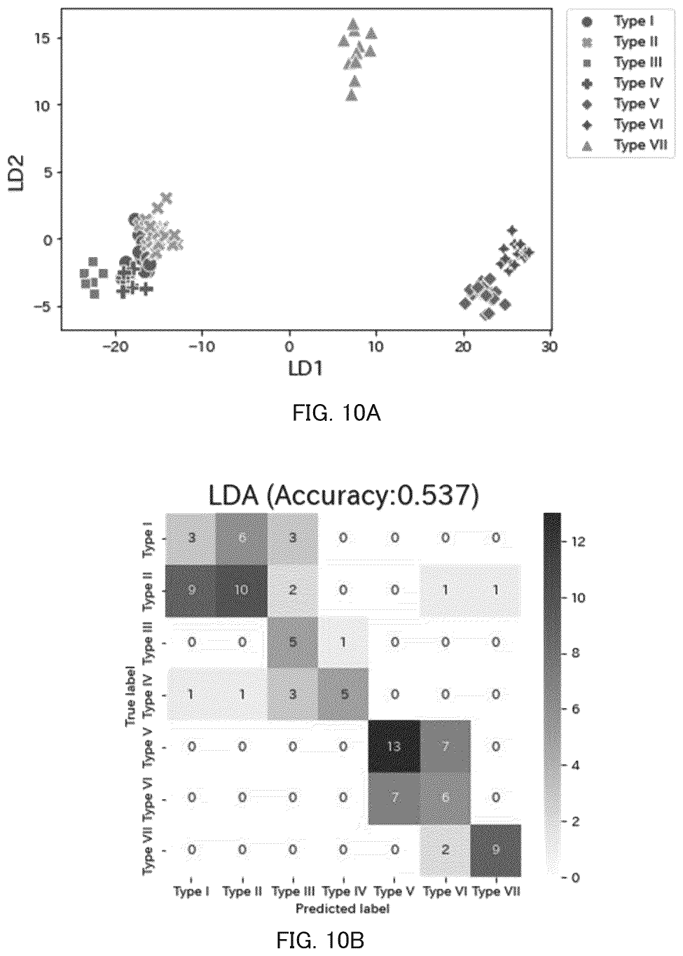

By using the luminescent dye molecules 1 and 32 to 45 (15 types), the steps from the luminescent dye molecule disposing step to the analyzing step described above were performed in a similar manner. The linear discriminant analysis model plot thus obtained is illustrated in FIG. 10A. and, six fold cross-validation was performed to calculate the accuracy rate and create a confusion matrix, and the generalization capability of the discriminant model was quantified. The confusion matrix is illustrated in FIG. 10B.

### Results

As illustrated in the 10B, according to the above-described discriminant model, 95 brands could be classified with an accuracy of about 54% for each type.

### (5) Analysis Using Luminescent Dye Molecules 46 to 60

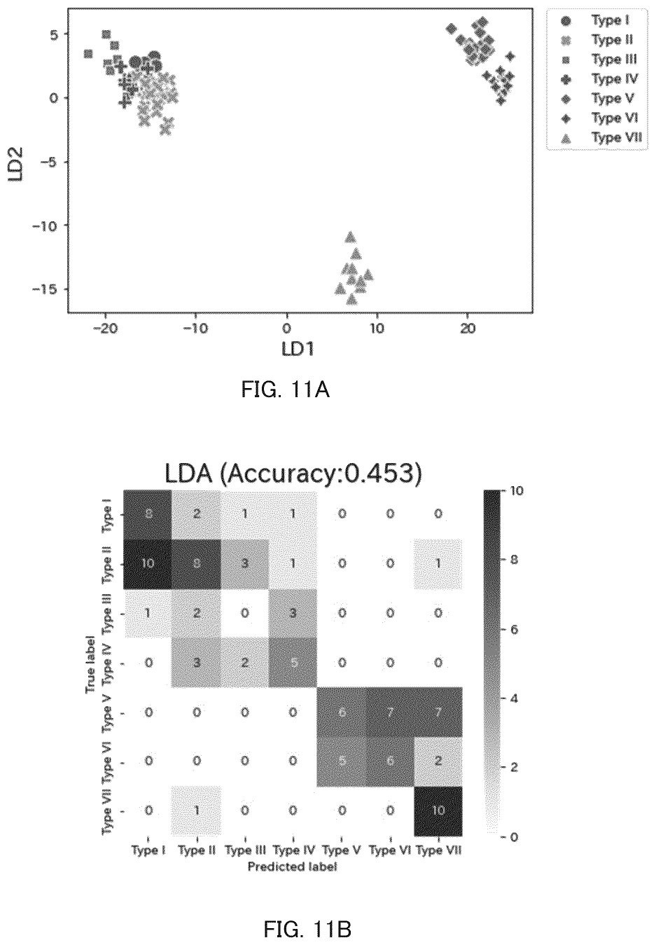

By using luminescent dye molecules 46 to 60 (15 types), the steps from the luminescent dye molecular disposing step to the analyzing step described above were performed in the same manner. The resulting linear discriminant model plot is illustrated in FIG. 11A. Similarly, the luminescent dye molecule disposing step to the second signal information acquiring step were performed. Then, calculation of a correct answer rate and creation of a confusion matrix were performed by cross-validation of six divisions, and generalization performance of the discriminant model was quantified. The confusion matrix is illustrated in FIG. 11B.

### Results

As illustrated in the 11B, according to the above-described discriminant model, 95 brands could be classified with an accuracy of about 45% for each type. The reason why the accuracy was lower than those of the other analyses may be from a small ratio of the β-form sugar structure in the luminescent dye molecule.

### (6) Analysis Using Luminescent Dye Molecules 61 to 75

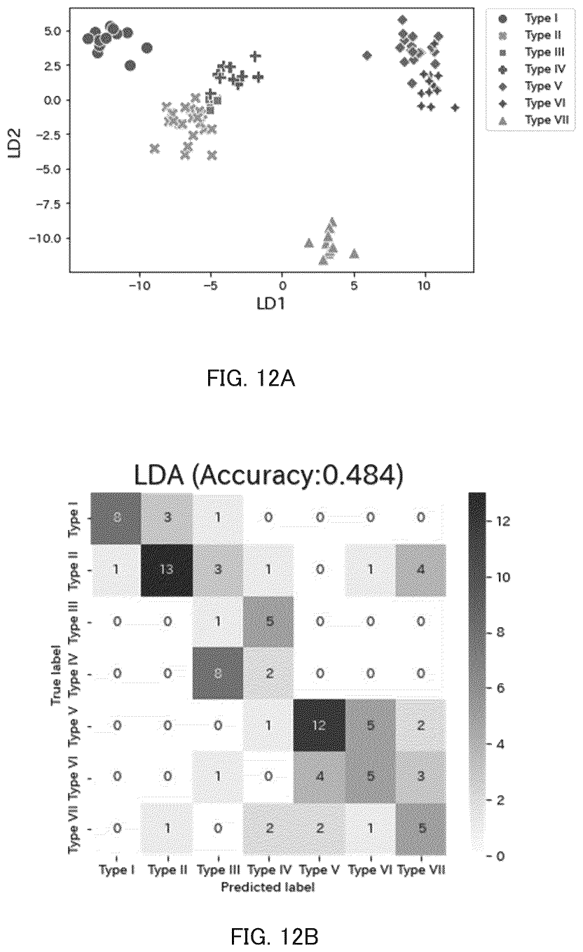

The luminescent dye molecules 61 to 75 (15 types) were used to perform the steps from the above-described luminescent dye molecular disposing step to the analyzing step in the same manner. The resulting linear discriminant model plot is illustrated in FIG. 12A. Then, calculation of a correct answer rate and creation of a confusion matrix were performed by cross-validation of six divisions, and generalization performance of the discriminant model was quantified. The confusion matrix is illustrated in FIG. 12B.

### Results

As illustrated in FIG. 12B, the discriminant model was able to classify the types of 95 brands of beverages with an accuracy of about 48%. This result shows that the present invention is applicable to peptide nucleic acids including a main chain structure different from that of natural DNA/RNA.

### (7) Analysis When Explanatory Variables are Randomly Replaced;

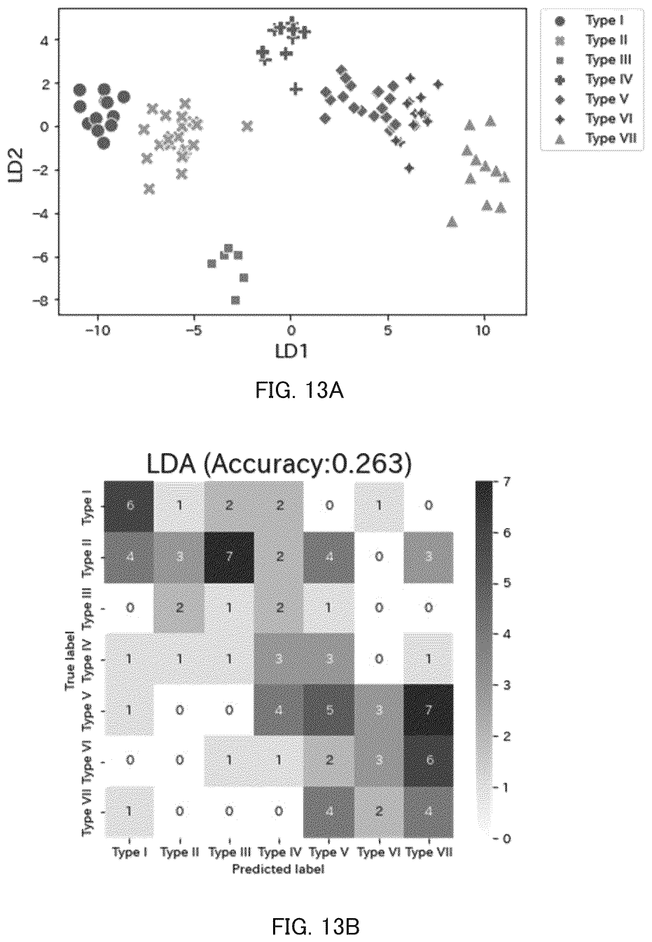

In order to clarify the effect of the present invention, a model was created by randomly replacing explanatory variables as a negative control experiment. To be specific, a discriminant model was created by randomly replacing explanatory variables obtained by performing the above-described measurement and analyzing steps using luminescent dye molecules 1 to 15 (15 types). The obtained linear discriminant analysis model plot is illustrated in FIG. 13A. Then, calculation of a correct answer rate and creation of a confusion matrix were performed by cross-validation of six divisions, and generalization performance of the discriminant model was quantified. The confusion matrix is illustrated in FIG. 13B.

### Results

As illustrated in FIG. 13B, the discriminant model in which the explanatory variables were randomly replaced was able to classify the types of 95 brands of beverages at about 25%. The results indicate that the above-described accuracy is not obtained by chance in a discriminant model in which the luminescent dye molecules are used and explanatory variables and an objective variable are correctly set. From the results, it is found that according to the discriminant model in which the luminescent dye molecules are used and explanatory variables and objective variables are correctly set, various compounds can be analyzed with high accuracy.

### 3. Example 3

The following monomers X1 to X13 were prepared by applying the monomer synthesis methods described in Example 1 and Example 2. In addition, when the above-described analysis method was performed using oligomers using these, discrimination was possible in the same manner as described above.

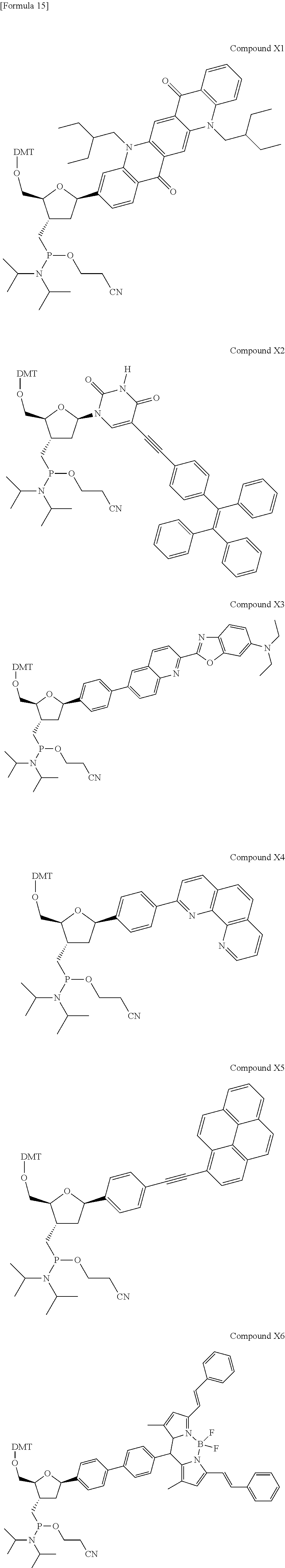

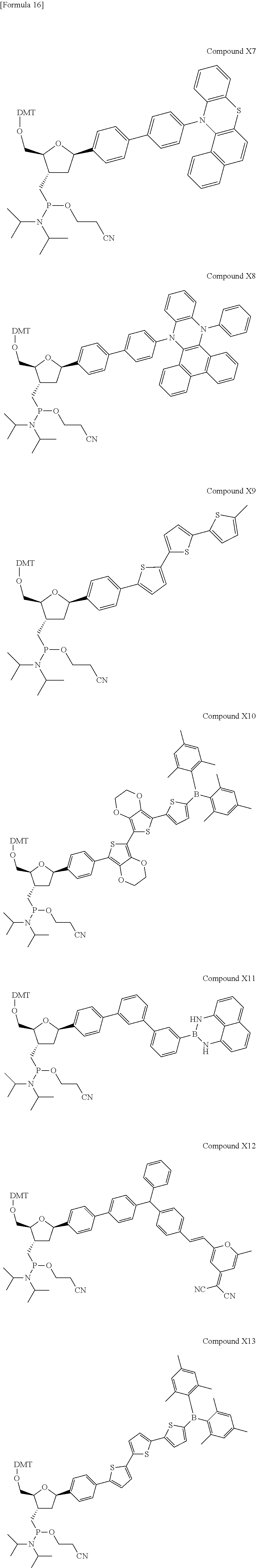

This application claims the benefit of Japanese Patent Application No. 2022-037179, filed Mar. 10, 2022, and Japanese Patent Application No. 2022-202044, filed Dec. 19, 2022. The contents described in the specification of the application and the drawings are all incorporated into {the specification of the present application.

### INDUSTRIAL APPLICABILITY

According to the above-described luminescent dye molecule, it is easy to interact with the target substance in the specimen, and a large amount of data can be acquired. Therefore, the present invention is very useful for analysis in various fields such as a medical field, an industrial field, and a food field.

Further, since the luminescent dye molecule or the carrier containing the luminescent dye molecule can be used as an indicator for inspection in the form of a solution or a dispersion, and a large amount of data can be obtained in a short time by utilizing an ink jet or an automatic dispenser, the present invention can greatly contribute to the activation and acceleration of industries such as data-driven research and development like inverse problem solving and data-driven inspection and diagnosis.

Furthermore, by using the fluorescent dye molecule of the present invention as an immobilized measurement chip, the portability is enhanced and the restriction on the place where it is used is almost eliminated.

The present invention can generate a large amount of real data highly compatible with machine learning and deep learning only by measuring light and color.

Furthermore, it is also characterized in that an expensive and large-sized instrumental analyzer is not required, and from the various characteristics as described above, it becomes possible to acquire data by bringing it to various working sites such as medical sites using liquid substances such as blood and saliva as a food source, food processing sites such as alcoholic beverages and fruit juice, production sites and water treatment plants in the chemical industry requiring sewage treatment, and further pastures for collecting milk and raw milk, so that it is expected to develop into a new technology which also matches the digital rural urban national concept proposed by the Japanese government.

1. A composite luminescent signal generating material for state sensing, the composite luminescent signal generating material comprising:
a nucleic acid structure; and
at least one luminescent compound residue bonded to a main chain of the nucleic acid structure, wherein
for a single excitation light, the composite luminescent signal generating material emits two or more types of light selected from the group consisting of fluorescence, phosphorescence, excimer light emission, exciplex light emission, thermally activated delayed fluorescence, excited state intramolecular proton emission, triplet triplet annihilation emission, twisted intramolecular charge transfer emission, and aggregation-induced emission.

2. The composite luminescent signal generating material according to claim 1, wherein
the nucleic acid structure is a structure derived from one or more compounds selected from the group consisting of DNA, RNA, phosphorothioate oligodeoxynucleotide, 2′-O-(2-methoxy) ethyl-modified nucleic acid, crosslinked nucleic acid, peptide nucleic acid, aTNA, SNA, GNA, LNA, and morpholino nucleic acid.

3. The composite luminescent signal generating material according to claim 1, wherein
the nucleic acid structure comprises
a main chain having at least one structural unit including
a sugar structure derived from pentose or hexose, and
a phosphate ester bond bonded to the sugar structure.

4. The composite luminescent signal generating material according to claim 3, wherein
50% or more of the sugar structure to which the luminescent compound residue is bonded is in a β-form.

5. The composite luminescent signal generating material according to claim 3, wherein
the at least one luminescent compound residue includes two or more luminescent compound residues, and the at least one structural unit includes two or more structural units.

6. The composite luminescent signal generating material according to claim 1, wherein
the composite luminescent signal generating material emits light in which the fluorescence, the excimer light emission, and the exciplex light emission are mixed.

7. The composite luminescent signal generating material according to claim 3, wherein:
the at least one luminescent compound residue includes two or more luminescent compound residues, and the at least one structural unit includes two or more structural units; and
the emitted light includes at least light derived from the phosphorescence or the thermally activated delayed fluorescence.

8. The composite luminescent signal generating material according to claim 3, wherein
the sugar structure is ribose or deoxyribose.

9. The composite luminescent signal generating material according to claim 3, wherein:
a signal generation portion containing the structural unit and the luminescent compound residue is linked with a base portion containing the structural unit and a nucleic acid base bonded to the structural unit, the nucleic acid base including a natural nucleic acid base; and
in the signal generation portion, a total number of the natural nucleic acid bases bonded to the sugar structure is 50% or less based on a total number of the sugar structures.

10. A luminescent substance carrier, comprising:
the composite luminescent signal generating material according to claim 1; and
a carrier particle carrying the composite luminescent signal generating material.

11. The luminescent substance carrier according to claim 10, wherein
the carrier particle comprises at least one of porous glass, porous silica gel, and/or polystyrene.

12. An ink for state sensing, the ink comprising:
the composite luminescent signal generating material according to claim 1; and
a solvent.

13. A measurement chip, comprising:
a plurality of the composite luminescent signal generating materials according to claim 1, wherein the plurality of composite luminescent signal generating materials are immobilized two dimensionally or three dimensionally.

14. A state sensing method, comprising:
generating a signal by causing a target substance and a plurality of the composite luminescent signal generating materials according to claim 1 to act on each other, and converting a state of the acting into an optical signal.

15. An analysis method comprising:
disposing one of a target substance and the composite luminescent signal generating material according to claim 1 into a reaction field configured to cause the target substance and the composite luminescent signal generating material to interact with each other, wherein the reaction field is in a plate;
acquiring first signal information from the plate in which the target substance or the composite luminescent signal generating material is disposed;
further disposing another one of the target substance and the composite luminescent signal generating material into the reaction field of the plate from which the first signal information has been acquired;
acquiring second signal information from the plate in which the target substance and the composite luminescent signal generating material are disposed; and
analyzing the first signal information and the second signal information by comparing the first signal information and the second signal information.

16. An analysis method comprising:
disposing one of a target substance and the luminescent substance carrier according to claim 10 into a reaction field configured to cause the target substance and the composite luminescent signal generating material of the luminescent substance carrier to interact with each other, wherein the reaction field is in a plate;
acquiring first signal information from the plate in which the target substance or the luminescent substance carrier is disposed;
further disposing another one of the target substance and the luminescent substance carrier into the reaction field of the plate from which the first signal information has been acquired;
acquiring second signal information from the plate in which the target substance and the luminescent substance carrier are disposed;
analyzing the first signal information and the second signal information by comparing the first signal information and the second signal information.

17. The analysis method according to claim 15, wherein:
the plate includes a plurality of the reaction fields different from each other; and
in the disposing of one of the target substance and the composite luminescent signal generating material or in the disposing of the other one of the target substance and the composite luminescent signal generating material, a plurality of types of the composite luminescent signal generating materials are disposed in the plurality of reaction fields, respectively.

18. An analysis method comprising:
putting one of a target substance and the composite luminescent signal generating material according to claim 1 into a fluorescence intensity measurement cell;
acquiring first signal information by using a fluorescence measurement device from the fluorescence intensity measurement cell in which the target substance or the composite luminescent signal generating material is put;
disposing another one of the target substance and the composite luminescent signal generating material into the fluorescence intensity measurement cell from which the first signal information has been acquired;
acquiring second signal information by using the fluorescence measurement device from the fluorescence intensity measurement cell in which the composite luminescent signal generating material and the target substance are disposed; and
analyzing the first signal information and the second signal information by comparing the first signal information and the second signal information.

19. The analysis method according to claim 15, wherein
in the acquiring of the first signal information and the acquiring of the second signal information, light having a predetermined wavelength is emitted to acquire light emission information.

20. The analysis method according to claim 15, further comprising
machine-learning the first signal information and the second signal information to create a learned model, wherein
in the analyzing of the first signal information and the second signal information, the learned model is referred to for performing the analyzing.
